(19) United States
(12) Patent Application Publication
(10) Pub. No.: US 2022/0267719 A1
(43) Pub. Date: Aug. 25, 2022

(54) METHOD FOR LOADING OF MICROORGANISMS ON MULTIPHASE BIOMATERIALS

(71) Applicant: Evonik Operations GmbH, Essen (DE)

(72) Inventors: Jürgen Erwin LANG, Karlsruhe (DE); Heike TOM DIECK, Friedrichsdorf (DE); Michael SCHWARM, Alzenau (DE); Bassam AL MESLMANI, Frankfurt am Main (DE); Dagmar FISCHER, Erlangen (DE)

(73) Assignee: Evonik Operations GmbH, Essen (DE)

(21) Appl. No.: 17/625,883

(22) PCT Filed: Jul. 10, 2020
(86) PCT No.: PCT/EP2020/069498

§ 371 Date: Jan. 10, 2022

(30) Foreign Application Priority Data
Jul. 12, 2019 (EP) 19186045.1

(51) Int. Cl. C12N 1/22; C12N 1/04; C12N 1/20; A61K 8/02; A61K 8/73; A61K 8/9728; A61K 8/99; A61L 15/28; A61L 15/36; A61P 31/04; A61P 17/10

(52) U.S. Cl. CPC C12N 1/22; C12N 1/04; C12N 1/20; A61K 8/0212; A61K 8/731; C12N 2523/00; A61K 8/99; A61L 15/28; A61L 15/36; A61P 31/04; A61P 17/10; A61K 8/9728

(57) ABSTRACT

The invention relates to method for loading microorganisms or parts thereof on and/or in pre-synthesized multiphase biomaterials comprising nanocellulose wherein the microorganisms are resuspended in a buffer or a culture medium and loaded into and/or onto the multiphase biomaterial and the use of such a loaded multiphase biomaterial in nutritional, food, pharmaceutical, medical, cosmetic, especially oral, mucosal, dermal and transdermal, ocular, dermatological or female health applications.

The present invention is directed to a method for loading microorganisms or parts thereof on and/or in pre-synthesized multiphase biomaterials comprising nanocellulose wherein the microorganisms are resuspended in a buffer or a culture medium and loaded into and/or onto the multiphase biomaterial and the use of such a loaded multiphase biomaterial in nutritional, food, pharmaceutical, medical, cosmetic, especially oral, mucosal, dermal and transdermal, ocular, dermatological or female health applications.

Probiotics are live microorganisms, which confer a health benefit on the host when administered in adequate amounts (FAO-WHO; Probiotics in food. Health and nutritional properties and guidelines for evaluation; FAO Food and Nutritional Paper 85, 2006). The most commonly investigated and commercially available probiotics are mainly microorganisms from species of genera Lactobacillus and Bifidobacterium. In addition, several others such as Propionibacterium, Streptococcus, Bacillus, Enterococcus, Escherichia coli, and yeasts are also used. Probiotic/synbiotic containing formulations such as supplements/cosmetics/biomedical/care products are systems “designed to have physiological benefits and/or reduce the risk of chronic disease beyond basic nutritional functions”.

The so-called prebiotics are defined as selectively fermented ingredients that results in specific changes in the composition and/or activity of the gastrointestinal microbiota, thus conferring benefits upon host health. Prebiotics often act as entrapping matrices during the gastrointestinal transit, further releasing the microorganism in the intestine and then serving as fermentable substrates (Koh et al., Food Microbiol. 2013 October; 36(1):7-13). Most prebiotics are complex carbohydrates from plant origin. Prebiotics and probiotics can be combined to support survival and metabolic activity of the latter and the resulting products belong to the class of synbiotics. Synbiotics refer to food ingredients or dietary supplements combining probiotics and prebiotics in a form of synergism, hence synbiotic (Pandey et al., J Food Sci Technol. 2015 December; 52(12):7577-87). According to the present invention the term synbiotics also includes synergistic combinations of probiotics with ingredients (“prebiotics”) creating metabolites with health benefits via selective metabolization of the ingredient by the added microorganism.

In this respect, probiotic bacteria arise as a valuable ingredient for dietary supplements, functional foods and topical applications suggested of being capable to support health and wellbeing. These are living microorganisms (in most cases), which are said to provide beneficial health effects to the host by replenishing natural microbiota, displaying regulatory properties by reducing pathogens by competition or by producing active metabolites at different locations (gut, skin, oral cavity, vaginal tract). However, probiotic bacteria when applied are very often inactivated by the conditions (e.g. harsh acidic stomach, bile acids or topical environmental factors etc.) and, consequently, the effectiveness of probiotic depends very much on the number of viable cells capable to reach the location of action. Thus, the development of smart delivery systems for cosmetic, biomedical or food applications, capable to entrap, protect, transport and appropriately deliver the active agent is important from a fundamental point of view for food applications, but also especially for topical applications.

Probiotics/synbiotics are well known for their health promoting beneficial effects at many locations of the mammalian/human subject e.g. gastrointestinal tract, skin, mucous membranes etc. One problem is to provide the beneficial probiotics/synbiotics to their location of action at required amounts, in active modus and for the required time to display an effect. With the latter aspects especially of importance for topical applications on skin and mucous membranes, such as inner and outer vaginal or oral mucous membranes. In many cases, for full beneficial and targeted action but also for survival of storage duration until use probiotics/synbiotics need additional co-factors and nutrients or starting materials and environmental requirements (like humidity etc.). Moreover, the combination with a specified ingredient/raw material can have a synbiotic effects with regard to the application. That rises additional challenge to the formulation of probiotics/synbiotics for topical applications. Formulation in creams for topical application often leads to only a transient availability and limited viability of bioactives.

Therefore, problems to be solved were to provide a simple and fast loading technique for probiotic microorganisms and providing a resulting product, which is able
to protect (often sensitive) probiotics/bioactives,
to reach high enough number for beneficial effects at the right (e.g. topical, gastrointestinal, vaginal) location,
to entrap/load bioactives, so that they are at location of action to reach sustained release of cells or actives/metabolites (prolonged action),
to allow for active ingredient production by the probiotics in situ, when combined with further ingredients, thus resulting in synbiotics,
to keep probiotics/synbiotics viable throughout storage until time of application,
to absorb the environmental fluid (e.g. tampon, oral application), and
to provide masking of potential bad odors.

Delivery systems for biomedical applications must address aspects of the entrapped biological as well as these relevant for the user. At best the delivery system has supportive effects itself, such as bacterial nanocellulose, with its low toxicity and, high water/fluid absorption capacity. The present invention provides as a solution a method that leads to a formulation in bacterial/microbial (nano)cellulose that protects probiotics and/or further bioactive ingredients for topical applications.

Nanocellulose is a term referring to nano-structured cellulose. This may be either cellulose nanocrystal (CNC or NCC), cellulose nanofibers (CNF) also called microfibrillated cellulose (MFC), or bacterial nanocellulose (BNC), which refers to nano-structured cellulose produced by bacteria. BNC is a nanofibrilar polymer produced by strains such as Komagataeibacter xylinus, one of the best bacterial species which given the highest efficiency in cellulose production. BNC is a biomaterial having unique properties such as: chemical purity, excellent mechanical strength, high flexibility, high absorbency, possibility of forming any shape and size due to extraordinary formability and softness and many others. Moreover, the material is vegetarian and vegan and comprises a high moisture content.

Production of BNC is becoming increasingly popular owing to its environmentally friendly properties. Many types of BNCs have been developed for various applications, including tissue regeneration, drug delivery systems, vascular grafts, and scaffolds for tissue engineering in vitro and in vivo (Czaja et al., Biomacromolecules 2007 January; 8(1):1-12; de Azeredo, Trends Food Sci Technol 2013 30:56-69; Almeida et al., Eur J Pharm Biopharm 2014 86:332-336; Oliveira Barud et al., Carbohydr Polym 2015 128:41-51; Martinez-Sanz et al., J Appl Polym Sci 2016133). Depending on the purpose of the application, BNC can provide improved mechanical qualities to the biomaterial owing to its biocompatibility, biofunctionality, lack of toxicity, and ease of sterilization (Klemm et al., Angew Chem Int Ed Engl 2011 50:5438-5466).

There are different formulations to deliver probiotics by usage of microbial cellulose, varying in use of additional polymers, immobilization/entrapment methods, resulting probiotic loading, viability, effectiveness/types of bacterial cells and general advantages such as handling, tolerability to the human intestine but none for topical applications. The survival time of probiotic bacteria should be within a certain limit not only while incorporated in a formulation. The known systems differ in providing protection to probiotic bacteria, but also in the dosage forms and survival rate at start of application. Actual loading techniques are mainly realized by time-consuming adsorption or by entrapment of microbial cells during microbial cellulose production. The lengthy incubation times have the disadvantage that the microorganisms are further growing during incubation and thereby the final loading concentration cannot be determined precisely.

The survival of probiotic lactic acid bacteria immobilized in different forms of BNC in simulated gastric juices and bile salt solution was analyzed, where immobilization of the microorganisms was performed by the adsorption of bacterial cells on the surface of the synthetized BNC and by a simultaneous cultivation of the probiotic bacteria with cellulose-synthetizing G. xylinus (Zywicka et al., Food Science and Technology 68, 2016, 322-328).

A comparative evaluation of bacterial cellulose (Nata) as a cryoprotectant and carrier support during the freeze process of probiotic lactic acid bacteria is described in a study, where bacterial cellulose produced by Acetobacterxylinum was compared for its cryoprotective and carrier support potential for probiotic lactic acid bacteria against other established cryoprotectants like 10% skim milk, calcium alginate encapsulation or 0.85% physiological saline and distilled water. Individual lactic acid bacteria were grown in MRS broth in the presence of nata cubes or the bacterial suspension mixed with either powdered bacterial cellulose (PBC), 10% skim milk, saline or distilled water and freeze dried, which resulted in a 3.0 log cycle reduction in the colony forming units as compared to the original cell suspension in the case of all the lactic acid bacteria (Bawa et al. Food Science and Technology 43, 2010, 1197-1203).

PL415670 discloses a method for immobilizing microorganisms on and/or in bacterial cellulose, which is characterized in that wet or dry bacterial cellulose is placed in suspension of Lactobacillus spp., 1° McFerland density, and is incubated in this suspension for 24 hours at room temperature 25° C. with shaking 180 rpm. For the immobilization of Lactbacillus spp. bacterial cellulose in the form of membranes or beads, obtained as a result of 6-day cultivation, respectively, under stationary conditions or shaking at 180 rpm, can be used. The immobilization method described in PL415670 allows for the immobilization of about 400×10⁵ cells of probiotic bacteria per gram of wet cellulose and obtaining the survival rate of bacteria immobilized on wet cellulose in the presence of simulated gastric acid above 50% and bile salts above 90% and immobilizing about 30×10⁵ cells of probiotic bacteria per gram of dry cellulose and obtaining the survival rate of bacteria immobilized on dry cellulose in the presence of simulated gastric acid above 50% and bile salts above 90%. However, the method described in PL415670 requires a long pre-cultivation of the immobilized bacteria and the bacterial cellulose material needs to be incubated in the bacterial suspension for 24 hours, which in summary is a time-consuming approach.

CN 109528691 A describes the production of microcapsules comprising cellulose nanofibers (CNF) and probiotics. The nanoparticles are prepared by mixing Lactobacillus plantarum with the solution comprising the nanofibers. This document reports a microencapsulation technique, where the delivery system (CNF) is formed during the loading process (formation and loading in one step). Therefore, the prepared CNF is blended with the probiotics in liquid and added dropwise to the crosslinker CaCl₂ solution to form the probiotics-cellulose nanofiber core. The produced core was then coated with alginate and chitosan applying the layer-by-layer method.

The disadvantages from the known techniques are lengthy procedures of loading (mainly lengthy adsorption time or co-cultivation), which may also lead to unwished and uncontrolled reproduction of probiotics. These long incubation times further leads to uncontrolled reduction of further ingredients and leads to non-uniform distribution of probiotics in the BNC network. The techniques known from the prior art are not fast and flexible with regard to the type of immobilized microorganisms.

The advantages of the present invention in view of the prior art are that the proposed method is a fast, simple and flexible/adaptable and cost-efficient method for loading of bacterial cellulose materials. The loading technique is very fast and controllable. It provides a sustainable resource saving method, using a quasi-inert carrier, which is natural and biocompatible. The resulting fleece structures comprise even 3D structure as well as high resistance and reduce transient probiotic availability by leading to sustained release and/or In-situ actives production. The present invention also allows very flat uniform or even transparent structure, or specifically shaped form.

Moreover, the present invention is very suitable for topical or intestinal applications (via oral) since probiotics/synbiotics are kept viable throughout storage until time of application onto the skin and especially while remaining on the skin. The products according to the present invention provide high liquid absorption capacity to absorb the (environmental) fluid (e.g. for tampon, pantry slips or oral application). Semi-dried systems are also suitable for the present invention. Moreover, potential bad odors can be masked with the product according to the present invention.

The present invention is related to a method for loading microorganisms or parts thereof on and/or in pre-synthesized multiphase biomaterials comprising nanocellulose, wherein the method comprises the following steps:
synthesizing a bacterially synthesized nanocellulose (BNC) multiphase biomaterial,
resuspending the microorganisms in a buffer or a culture medium and
loading the microorganisms into and/or onto the BNC material by either
a) mixing the multiphase biomaterial with the microorganisms at 300 rpm or more, preferably between 500 rpm and 3.500 rpm, for 1 to 60 min, preferably between 5 and 10 min, at a temperature of 37° C. or less, preferably between 10° C. and 37° C., or
b) injecting the microorganisms into the multiphase biomaterial and incubating at a temperature of 37° C. or less, preferably between 4° C. and 37° C. for up to 72 h, preferably for up to 1 h, or
c) incubation of the multiphase biomaterial in the buffer or culture medium with resuspended microorganisms at a temperature of 37° C. or less for 60 min or less, preferably 10 min or less.

The incubation time in step a) is between 1 and 60 min, preferably between 5 and 10 min. The process of step a) is also named “high speed method” for the present invention and is achieved by using a vortexer. In a preferred configuration, the BNC non-woven is vortexed (Vortex Genie 2) together with a bacterial suspension at vortex strength 10.5 (˜3300 rpm) at room temperature for 10 min. The loading suspension is then removed, the BNC non-woven is washed under vortex for 10 sec.

In step b) the incubation time is for up to 72 h, preferably for up to 1 h. In general, for the injection method according to b) no long incubation times are necessary for loading of the microorganisms. Therefore, in a preferred embodiment, the incubation time is between 1 s and 1 h, preferably between 1 sec and 10 min, more preferably between 1 sec and 60 sec.

A method for synthesizing a bacterially synthesized nanocellulose (BNC) multiphase biomaterial is disclosed in US 2015/0225486.

It is preferred to use a non-woven BNC material, as described in WO 2018 215598 A1 for example. According to the present invention, a non-woven BNC material is in particular a non-woven of fibers of BNC. The terms “non-woven BNW” and “BNC fleece” may be used interchangeably in accordance with the present invention.

In a preferred embodiment, the microorganisms are sprayed onto the multiphase biomaterial. In a preferred embodiment a bacterial suspension or a bacterial powder is sprayed in a preferred configuration. It is preferred to spray a bacterial suspension onto the BNC non-woven for 1 min or less.

In an advantageous configuration of the method for loading microorganisms or parts thereof on and/or in pre-synthesized multiphase biomaterials comprising nanocellulose, loading of the microorganisms into and/or onto the BNC material is achieved by either:
a) mixing the multiphase biomaterial with the microorganisms at 300 rpm or more, preferably between 500 rpm and 3.500 rpm, for 1 to 60 min, preferably between 5 and 10 min, at a temperature of 37° C. or less, preferably between 10° C. and 37° C., or
b) injecting the microorganisms into the multiphase biomaterial and incubating at a temperature of 37° C. or less, preferably between 4° C. and 37° C. for up to 72 h, preferably for up to 1 h.

With options a) and b) the microorganisms are better distributed among the multiphase biomaterial and the microorganism are attached more thoroughly to the multiphase biomaterial.

The present invention relates to a process for the loading of bacteria using bacterial cellulose which is a component of the carrier for temporarily immobilizing bacteria, where the temporarily immobilized bacteria offer beneficial effects in topical applications (e.g. on skin or mucous membranes). It provides a method to gain a formulation that incorporates/entraps/temporarily immobilize/load probiotics/synbiotics in bacterial cellulose for topical application in cosmetics, biomedical or personal care providing e.g. transdermal, anti-inflammatory, calming, anti-wrinkle/anti-aging, pathogen-inhibiting or regulating, acidificating, anti-redness, or other appearance-promoting effects. Examples are amongst others probiotic/synbiotic masks, patches, panty liners, tampons etc.

The carrier functions as habitat for the probiotics/synbiotics. The therein-immobilized biologicals are used for the triggered biosynthesis and release of metabolites, enzymes or release of bacteria cells itself for beneficially influencing the respective topical environment (e.g. skin, oral, vaginal).

The bacterial cellulose is a three-dimensional network and is the carrier to immobilize and trap the microorganism and further substances. The immobilized biologicals (including the microorganisms) are used for the biosynthesis of bioactive metabolites (e.g. antimicrobials, metabolic bioactive) in situ/in vivo, triggered release of the microorganisms and bioactives and/or used as immobilized microfactories for fermentation processes.)

Application areas might be cosmetics (improved appearance e.g. of redness in Rosacea or Acne), but also medical application (vaginal dysbiosis) and hygienics for women or other consumer goods.

In a preferred embodiment, the microorganisms are loaded as vegetative cells or in a dormant form, preferably as bacterial spores, or as a cell-extract. In an advantageous configuration of the present invention, the microorganisms are dried, preferably spray-dried or freeze-dried and used in a powder form.

Many bacteria can survive adverse conditions such as temperature, desiccation, and antibiotics by endospores, exospores (microbial cysts), conidia or states of reduced metabolic activity lacking specialized cellular structures. Up to 80% of the bacteria in samples from the wild appear to be metabolically inactive, many of which can be resuscitated. Such dormancy is responsible for the high diversity levels of most natural ecosystems. An endospore is a dormant, tough, and non-reproductive structure produced by certain bacteria from the phylum Firmicutes. Endospore formation is usually triggered by a lack of nutrients, and usually occurs in gram-positive bacteria. In endospore formation, the bacterium divides within its cell wall, and one side then engulfs the other. Endospores enable bacteria to lie dormant for extended periods, even centuries. When the environment becomes more favorable, the endospore can reactivate itself to the vegetative state. Most types of bacteria cannot change to the endospore form. Examples of bacteria that can form endospores include Bacillus and Clostridium. The endospore consists of the bacterium's DNA, ribosomes and large amounts of dipicolinic acid, a spore-specific chemical that appears to help in the ability for endospores to maintain dormancy and accounts for up to 10% of the spore's dry weight.

In alternative configurations of the present invention, the microorganisms are wet or dry and/or pre-cultured or not pre-cultured. The multiphase biomaterial is wet or dried or partially dried or re-swelled in buffer.

It is preferred, when the nanocellulose is derived from a plant, algae or a microorganism, preferably from Komagataeibacter, more preferably Komagataeibacter xylinus. Komagataeibacter xylinus is a species of bacteria best known for its ability to produce cellulose. It has since been known by several other names, mainly Acetobacterxylinum and Gluconacetobacterxylinus. It was given its current name, with the establishment of the new genus Komagataeibacter, in 2012.

For the present invention, it is preferred to use BNC non-woven with an average thickness of at least 0.5 mm for loading of the microorganisms. It is particularly preferred, when the BNC non-woven has an average thickness of between 1 mm and 5 mm, more preferably between 2 mm and 3 mm. It could be shown that a better re-swellability of the loaded BNC non-woven could be achieved, when the BNC non-woven had an average thickness of between 2 mm and 3 mm.

In a specific embodiment of the present invention, the nanocellulose is bacterially synthesized nanocellulose (BNC) comprises a layered structure, which is preferably selected from
BNC comprising a network of cellulose fibers or nanowhiskers,
BNC comprising two or more different layers of cellulose fibrils, wherein each layer consists of BNC from a different microorganism or from microorganisms cultivated under different conditions,
BNC comprising of at least two different cellulose networks or
a BNC composite material further comprising a polymer.

Cellulose nanowhiskers (NW), also known as cellulose nanocrystals or nanocrystalline cellulose, present an important nanoscaled material that holds great promise in different applications (Råanby et al., Acta Chem Scand 3, 649-650, 1949). NW are a result of the incomplete degradation of cellulose (Plötzinger et al., Cellulose 25, 1939-1960, 2018).

In an advantageous embodiment of the present invention, at least two different bacterial cellulose networks are designed as a combined homogenous phase system or as a layered phase system consisting of at least one combined homogenous phase as well as at least one single phase, preferably in combination with further polymers.

A preferred method is described in EP2547372. It is particularly preferred, when at least two different cellulose-producing bacterial strains are prepared together or separated are synthesized together to several different bacterial cellulose networks in a common culture medium, wherein the BNC structure and the BNC properties of the multiphase biomaterials are affected by the choice of the at least two different bacterial strains, by their preparation and inoculation as well as by influencing the synthesis conditions, wherein the bacterial cellulose networks are synthesized as a combined homogenous phase system or as a layered phase system consisting of at least one combined homogenous phase as well as at least one single phase. Moreover, it is preferred, when the at least two different bacterial cellulose networks are prepared independently from each other and are subsequently brought together and are synthesized together. In an advantageous configuration for the conjoint synthesis the at least two different bacterial cellulose networks are brought together already before the inoculation.

In a preferred configuration of the present invention, further substances are added during bacterial synthesis of BNC that allow to control the resulting pore/mesh sizes, preferably selected from polyethylene glycol (PEG), β-cyclodextrin, carboxymethyl cellulose (CMC), methyl cellulose (MC) and cationic starches, preferably selected from 2-hydroxy-3-trimethylammoniumpropyl starch chloride and TMAP starch.

This modification allows specifically tailoring the BNC for the microorganism to be loaded. As a remarkable benefit of bacterial cellulose, the property-controlling fiber network and pore system formed by self-assembly of the cellulose molecules can be modified in situ using additives during biosynthesis. This allows to adapt the pore size to the size of the microorganisms, which are to be loaded. The addition of polyethylene glycol (PEG) 4000 causes a pore size decrease. In presence of β-cyclodextrin or PEG 400 remarkably increased pores can be achieved. Surprisingly, these co-substrates act as removable auxiliaries not incorporated in the BC samples. In contrast, carboxymethyl cellulose and methyl cellulose as additives lead to structural modified composite materials. Using cationic starch (2-hydroxy-3-trimethylammoniumpropyl starch chloride, TMAP starch) double-network BC composites by incorporation of the starch derivative in the BC prepolymer were obtained (Hessler & Klemm, Cellulose 16(5):899-910, 2009).

In an advantageous configuration of the present invention, in a subsequent step the loaded multiphase biomaterials are incubated with a moisture binder for drying, wherein the moisture binder is an osmotically and/or hygroscopically effective solution, preferably containing single saccharides, salts, saccharide-containing or saccharide-like substances, polyethylene oxides, a combination of different representatives of these moisture-binding groups of substances and/or a combination of one and/or more representatives of these moisture-binding groups of substances with one or more surfactants and/or one or more preservatives.

The moisture binder is added for the purpose of drying and preserving the swellability with almost complete reconstitution of the cellulose structure and consistency is subjected to the adsorbent effect of a moisture binder and after this adsorbent exposure is dried regardless of any structural change to the material. A process for drying is described in WO02013060321A2.

As a moisture binder an osmotically and/or hygroscopically effective solution is used, preferably containing single saccharides, salts, saccharide-containing or saccharide-like substances, polyethylene oxides, a combination of different representatives of these moisture-binding groups of substances and/or a combination of one and/or more representatives of these moisture-binding groups of substances with one or more surfactants and/or one or more preservatives. Moisture binders, which are preferably used are glucose, magnesium chloride, saccharide. In a preferred configuration, for further modification of the reswelling behavior in addition to the moisture binder a surfactant and/or preservative-containing solution is used.

The moisture-binding solution can have a concentration of osmotically active and/or hygroscopic substances of 0.01% up to the saturation limit, preferably of 5-20%. It is preferred to use the surfactants and/or preservatives which are used in combination with the osmotically and/or hygroscopically effective solution in a concentration of 0.01% up to the saturation limit, preferably of 0.01-10%.

The cellulose or the cellulose-containing material being treated with the moisture binder can be air-dried, or vacuum-dried.

The cellulose or the cellulose-containing material to be subjected to the adsorbent effect of the moisture-binding solution is dipped into the moisture-binding solution in a preferred configuration. In an alternative configuration, the moisture-binding solution is sprayed, dropped, brushed or cast onto the cellulose or the cellulose-containing material to be subjected to the adsorbent effect of the moisture-binding solution. Alternatively, the moisture binder is already added in addition to the cellulose cultivation process for the purpose of its adsorbent exposure.

In a preferred embodiment, the microorganism is a probiotic bacterial or yeast strain selected from Bifidobacterium, Carnobacterium, Corynebacterium, Cutibacterium, Lactobacillus, Lactococcus, Leuconostoc, Microbacterium, Oenococcus, Pasteuria, Pediococcus, Propionibacterium, Streptococcus, Bacillus, Geobacillus, Gluconobacter, Xanthonomas, Candida, Debaryomyces, Hanseniaspora, Kluyveromyces, Komagataella, Lindnera, Ogataea, Saccharomyces, Schizosaccharomyces, Wickerhamomyces, Xanthophyllomyces and Yarrowia, preferably Cutibacterium acnes, Lactococcus lactis, Lactobacillus rhamnosus, Lactobacillus crispatus, Lactobacillus gasseri, Bacillus subtilis, Bacillus megaterium, Micrococcus luteus, Micrococcus lylae, Micrococcus antarcticus, Micrococcus endophyticus, Micrococcus flavus, Micrococcus terreus, Micrococcus yunnanensis, Arthrobacter agilis, Nesterenkonia halobia, Kocuria kristinae, Kocuria rosea, Kocuria varians, Kytococcus sedentarius, Dermacoccus nishinomiyaensis or mixtures thereof.

It is further preferred to use S. epidermidis, L. fermentum, DSM 32609 L. rhamnosus, DSM 32758 L. plantarum, DSM 32749 L. delbrueckii susp. bulgaricus, DSM 33370 L. plantarum LN5, DSM 33377 L. brevis LN32, DSM 33368 L. plantarum S3, DSM 33369 L. plantarum S11, DSM 33376 L. paracasei S20, DSM 33375 L. paracasei S23, DSM 33374 L. reuteri F12, DSM 33367 L. plantarum F8, DSM 33366 L. plantarum S4, DSM 33364 L. plantarum S28, DSM 33363 L. plantarum S27, DSM 33373 L. paracasei S18a, DSM 33365 L. plantarum S18b, DSM 33362 L. plantarum S13, DSM 32767 Lactococcus lactis sups. lactis, L. fermentum DSM 32750, Propionibacterium acnes, Cutibacterium acnes.

In a further preferred embodiment of the present invention, an additional step is performed before or after or in parallel to loading of the multiphase biomaterials with the microorganisms, wherein the multiphase biomaterials are loaded with further ingredients and/or nutrients selected from amino acids, fatty acid salts, anthocyanins, monosaccharides and extracts, preferably lysine salt of DHA and EPA, rhamnose, tryptophan. These further ingredients may provide metabolites with health benefits derived from metabolization by the microorganisms or can selectively be fermented by the microorganisms and can be classified as prebiotics. Such a composition comprising the probiotic microorganism and one or more ingredients/prebiotics as defined above can be named as synbiotic.

A further aspect of the present invention is directed to a non-woven multiphase biomaterial comprising nanocellulose consisting of at least two different bacterial cellulose networks comprising at least one living microorganism obtainable by a method according to the present invention.

In an advantageous configuration, the multiphase biomaterial comprises at least one living microorganism at a concentration of at least 3.00×10⁷ cells of microorganism per gram of cellulose.

The present invention is also directed to the use of a non-woven multiphase biomaterial according to the present invention in food, oral, mucosal, dermal and transdermal, ocular, nutritional, cosmetic, dermatological, oral or female health applications.

Such cellulose products, for example, relate to the use in medical (z. B. implant material, wound dressings, skin substitute material), pharmaceutical (for example, drug delivery systems) and technical applications (such as filter and membrane systems).

Therefore, cellulose and cellulose-containing materials may be provided with a minimum of time and technical-economic costs without damaging stress on the cellulose and without stability and loss of effect of any loaded materials, such as drugs, probiotics, additives, etc.

### WORKING EXAMPLES

### Example 1: Incorporation of Probiotics without Using Additional Polymer (after Pre-Culture)

A) Characterization and Sterilization of the BNC Before Loading Regarding Dimensions (Surface, Volume, Thickness, Weight)

All BNC fleeces were stored at 4° C. (or at room temperature when packed) and were equilibrated to room temperature for 30 min. Diameter and height were measured using the Vernier caliper scale at 3 different locations of the fleece. The mean values and standard deviation of diameter and height as well as of the volume (V) of the BNC fleeces were calculated using the following formula 1:

V=πr²h (1)

with π=3.14, r: radius, h: height.

Furthermore, the surface area (A) of each BNC fleece was determined applying the formula 2:

A=2πrh+2πr² (2)

with π=3.14, r: radius, h: height.

The data were expressed as mean±standard deviation of all measurements.

The characterization of the BNC fleece dimensions was carried out for the standard BNC fleeces synthesized according to the standardized method of the local laboratory. The BNC fleeces demonstrated a weight of 1.16±0.06 g, a diameter of 1.6±0.07 cm and a height of 0.5±0.04 cm. A surface area of 7.24±0.27 cm² was detected for each BNC fleece at a volume of 1.2±0.1 cm³.

Thin BNC fleeces for application as mask or patch or in rolled form are characterized by a thickness of 1-4 mm at best a thickness of 2-3 mm height to ensure optimal re-swellability.
B) Loading of BNC Fleeces with Probiotic Suspension

### Preparation of Probiotics Cultures and Suspensions (L. lactis, B. Subtilis)

Under sterile conditions, 2 sterilized 100 ml glass Erlenmeyer flask were filled with 20 ml sterilized MRS broth bouillon at pH 6.2±0.2 for L. lactis. 2 sterilized 100 ml glass Erlenmeyer flask were filled with 20 ml sterilized TSB medium at pH 7-7.2 for B. subtilis. About 2 mg of L. lactis powder were added to the MRS medium and mixed, where one flask was prepared with the probiotic strain and one with MRS blank. Subsequently, 5 μl of the B. subtilis cryo suspension were added to the TSB medium and mixed. One flask was prepared with the probiotic strain and one with TSB blank. The probiotic cultures were prepared under sterile conditions and incubated at 37° C. under shaking at 100 rpm for 8 h; the control MRS medium was incubated under the same conditions. After 8 h, the cultures were transferred from the incubator to the laminar air flow bench, mixed and 500 μl of each culture were collected in a sterilized 2 ml Eppendorf cup using a sterilized 1 ml pipette. The optical density (OD₆₀₀) of the collected samples was measured three times for each at a wavelength of 600 nm in comparison to the blank MRS or TBS medium using a UV cuvette and optical density spectrophotometer (Biophotometer). The volume to prepare 10 ml probiotics suspension at concentration of OD₆₀₀ 0.5=10⁸ cells/ml (loading ratio=1 g BNC: 10 ml loading solution) was calculated and the last calculated volume was filled into 50 ml sterilized tube and the total volume was completed up to 10 ml using the corresponding medium (MRS for L. lactis and TSB for B. subtilis) or saline NaCl 0.9% and mixed afterwards.

I. Loading of BNC Fleeces with Probiotics Suspension by High Speed Method (Vortex)

The BNC fleeces were added to the probiotic suspension in 50 ml tubes (L. lactis, B. subtilis). The control BNC fleeces were added into sterilized medium or saline. The tubes were vortexed (Vortex Genie 2) at vortex strength 10.5 (˜3300 rpm) at room temperature for 10 min. The loading suspension was removed, and the BNC fleeces were washed in 10 ml saline under vortex for 10 sec.

II. Loading of BNC Fleeces with Probiotic Suspension by Injection Method

The BNC fleeces were prepared as described above. The probiotic suspension was prepared at a concentration of 10⁸ cell/125 μl. The syringe needle was inserted into the center of the BNC fleeces and the volume was injected (5 units).

III. Loading of BNC Fleeces with Probiotic Suspension by Spraying (Pre-Cultured and not Pre-Cultured)

The BNC fleeces were prepared as described above. 10 ml of probiotic suspension (pre-cultured) in saline were prepared of each L. lactis and B. megaterium at concentration OD₆₀₀ 0.5=10⁸ cells/ml. 5 ml of the probiotics suspension was sprayed homogeneously on the BNC (mask patch or other form) using the sterilized glass reagent sprayer (Sterilized glass reagent sprayer. Art Nr: 11526914. Fischer scientific, Germany). For loading of probiotics in powder form procedure is as follows: Under sterile conditions in laminar air flow bench (Heraeus HS 18/2), 100 mg of the Probiotic e.g. (L. lactis) powder were weighted in sterilized 2 ml Eppendorf using the balance (Sartorius H95 Basic). The L. lactis powder was directly sprayed on the BNC fleeces applying compressed air. Alternatively, a probiotic suspension (e.g. L. lactis) was prepared with the powder form under sterile conditions in laminar air flow bench in MRS broth medium and saline at concentration of OD₆₀₀: 1 McFarland by adding the L. lactis powder into 35 ml of MRS or saline in 50 ml centrifuge tubes and mixing. The L. lactis powder-suspension was then sprayed onto the BNC fleeces using the sterilized glass reagent sprayer (Sterilized glass reagent sprayer. Art Nr: 11526914. Fischer scientific, Germany).

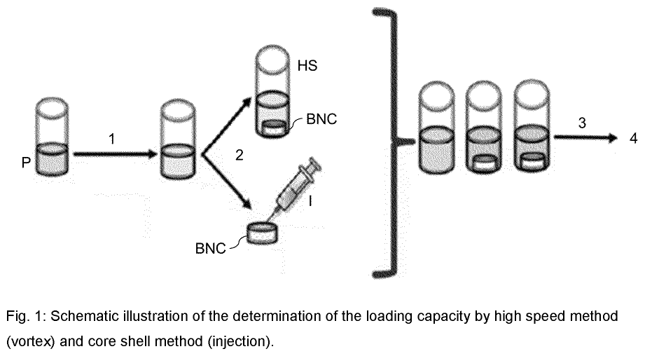

An overview over the different loading techniques is shown in FIG. 1: Schematic illustration of the determination of the loading capacity by high speed method (vortex) and core shell method (injection). A probiotic culture (P) was centrifuged and resuspended in saline NaCl 0.9% at OD₆₀₀ 0.5 (step 1) and loaded onto BNC by either high speed method (HS) (3300 rpm, 10 min, 22° C.) or direct injection (I) (125 μl, OD600 0.5) (step 2). The loaded BNC and control probiotics were re-cultured for 18 h at 37° C. and 100 rpm (step 3) and OD₆₀₀ was determined subsequently (step 4).

### Example 2: Loading Capacity by Vortex and Injection Loading Method of BNC Fleeces with Probiotics Suspensions (L. lactis, B. Subtilis) (with Pre-Culturing)

A) Characterization of Loading: Loading Capacity, Location, Homogeneity of Distribution

The probiotics cultures were incubated for 8 h at 37° C. and 100 rpm shaking. Afterwards, they were centrifuged at 4000 rpm for 10 min and resuspend in saline NaCL 0.9%. The OD₆₀₀ was adjusted to 0.5=10⁸ cells/ml saline. The BNC were loaded with the probiotic cultures by vortex or injection method as described for example 1 under BI and BII. A schematic illustration of the determination of the loading capacity by high speed method (vortex) and core shell method (injection) is shown in FIG. 1. BNC fleeces were loaded with probiotics at OD₆₀₀ of 0.5 McFarland (corresponding to ˜10⁸ cells/ml) before they were re-cultured in growth medium at 37° C. and 100 rpm for 18 h. The loading capacity was determined by measuring the OD₆₀₀ of the recultured BNC in comparison to the OD₆₀₀ of a standard probiotic culture prepared by adding the same quantity of probiotics OD₆₀₀ 0.5 McFarland (corresponding to ˜10⁸ cells/ml) to the growth medium. In microbiology, McFarland standards are used as a reference to adjust the turbidity of bacterial suspensions so that the number of bacteria will be within a given range to standardize microbial testing.

The quantity of loaded probiotics is a decisive factor determine the efficiency of the developed form and define to same extent the activity of probiotics. The number of loaded probiotics was investigated to assess the loading capacity of the employed procedures and to measure the number of released probiotics from loaded BNC fleeces. The loading process was carried out in isotonic solution to inhibit the proliferation of probiotics during the experiment. The probiotic loaded BNC fleeces were re-cultured in the appropriate medium in comparison to free probiotics cultured under the same conditions and concentrations.

The loading capacity of B. subtilis by high speed method (vortex) and core shell method (injection) was determined. The BNC fleeces were loaded with probiotics at OD₆₀₀ 0.5 McFarland (corresponding to ˜10⁸ cells/ml) before they were re-cultured in TSB growth medium at 37° C. and 100 rpm for 8 h. The loading capacity was determined by measuring the OD₆₀₀ of the re-cultured BNC in comparison to the OD₆₀₀ of a standard B. subtilis culture prepared by adding the same quantity of OD₆₀₀ 0.5 McFarland (corresponding to ˜10⁸ cells/ml) to the growth medium. A turbidity in the bottles of probiotic loaded BNC fleeces was obvious indicating the release and proliferation of the probiotics from the BNC fleeces into the culture medium. Both probiotics exhibited a higher loading capacity by the injection method compared to the high-speed method. L. lactis demonstrated a loading capacity of 10.1%±2.2% by the vortex method compared to 36.2%±4.7% by injection method. B. subtilis exhibited a loading capacity of 22.14%±3.1% by the vortex method and 42.85%±5.4% by the injection method.
B) Detection of Loading Location by Autofluorescence of the Microorganisms

### Preparation of Live/Dead Stain Solution

The Live/Dead BacLight Bacterial viability kit L7012 was prepared according to the manufacturer's instructions.

For L. lactis and B. subtilis, the volume of probiotics culture to prepare 50 ml suspension at OD₆₀₀=0.5 was calculated and the last volume was centrifuged at 4000 rpm at room temperature for 15 min. The pellet was resuspended in 1 ml purified water and 1 ml of the last prepared Live/Dead stain solution was added, mixed and incubated at room temperature in dark for 15 min. After 15 min, the stained probiotics were centrifuged at 4000 rpm at room temperature for 10 min. The stain solution was removed, and the stained probiotics were re-suspended in 30 ml sterilized saline and vortexed for 10 sec to wash the stained probiotics. The re-suspended probiotics were centrifuged at 4000 rpm at room temperature for 10 min and re-suspended in 50 ml sterilized saline.

Visualization of the Probiotic Distribution in the BNC Fleeces

The BNC fleece was transferred into 50 ml tubes and 5 ml methylene blue stain was added at concentration of 1% and kept at room temperature for 10 min. The methylene blue solution was removed, and the BNC fleece was washed three times under vortex with 30 ml saline for each. Afterwards, the methylene blue-stained BNC fleeces were loaded with the Live/Dead stained probiotics in saline by vortex method. As a control, methylene blue stained-BNC fleeces were immersed in 10 ml saline and mixed under the same conditions. The loading suspensions were removed, and the BNC fleeces were washed in 10 ml saline under vortexing. The fleeces were illuminating in top view and cross sections with the Moleculight and photographs were taken.

The distribution of the probiotics in the BNC fleeces was detected by applying a fluorescence staining method. The BNC was stained with methylene blue to eliminate its auto fluorescence. The live/dead-stained probiotics were then incorporated into the BNC fleeces by vortex and injection method and detected using a fluorescence detecting camera. The photographs of the top and the cross sections indicated that L. lactis was homogeneously distributed throughout the whole cross section with only a slight trend to the pre polymer which can uptake more material due to its looser structure with larger pores. B. subtilis revealed a strong tendency to incorporate into the pre polymer which might be related to its larger germ size.

Evaluation of Loading Homogeneity and Distribution by Scanning Electron Microscopy (SEM)

The loaded BNC fleeces were fixed and dried using critical point drying before they were sputter coated and observed by scanning electron microscopy (SEM). The subsequence procedure was performed as follows: The BNC fleeces were fixed in 3 ml/well fixing solution of 2.5% glutaraldehyde and 4% formaldehyde in sodium cacodylate buffer M, pH 7.4 at room temperature for 10 h. Afterwards, the fixing solution was removed and the BNC fleeces were washed three times in saline before a dehydration process in an ethanol series at increasing concentrations (30%, 50%, 70%, 80%, 90%, 100% and 100%) was completed for 15 min each. The BNC fleeces were dried by critical point drying in a Leica EM CPD300 Automated, Critical point dryer (Leica). BNC pieces were then mounted onto a SEM sample holder and sputter coated with gold (layer thickness 30 nm) in a sputter coater (BAL-TEC SCD005 Sputter Coater) under vacuum using an inert gas (argon) before they were analyzed and microscopically imaged using a Sigma-VP-scanning electron microscope (Carl Zeiss, Germany), operated at 5 kV using the In-lens-detector.

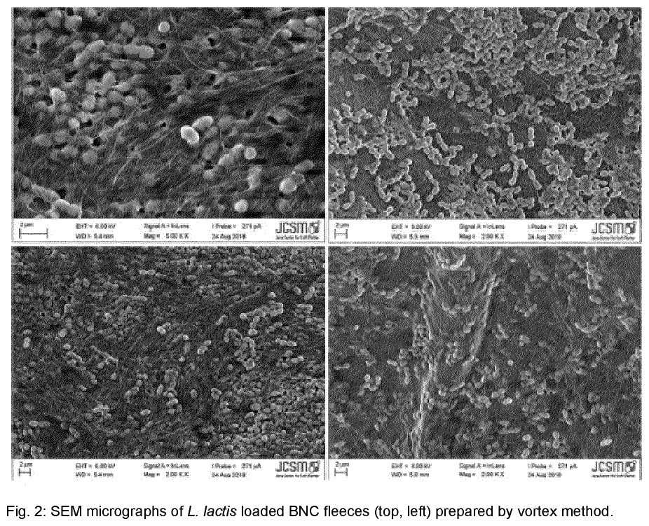

The distribution of the probiotics in BNC fleeces after loading by the vortex method was determined by scanning electron microscopy (SEM) in comparison to native non-loaded BNC fleeces at different sections. Both the non-loaded and the probiotics loaded BNC fleeces were fixed in a mixture of glutaraldehyde and formaldehyde to stabilize the final form and maintain the location of the loaded probiotics before drying and SEM imaging were completed. The microscopic analysis of the BNC fleeces showed a widespread distribution of the loaded probiotics on the surface of the BNC fleeces as demonstrated in FIG. 2. Furthermore, the loaded probiotics were homogenously localized on the cross and vertical sections confirming the homogeneity of loading inside the BNC fleeces applying vortex method.

FIG. 2 shows SEM micrographs of L. lactis loaded BNC fleeces (top, left) prepared by vortex method. The L. lactis loaded BNC fleeces were inspected at different sections; on the surface (top, right), on the cross section (bottom, right) and vertical section (bottom, left). Micrographs were taken at 5 kV using the in-lens-detector at the magnification.

### Example 3: Loading of BNC Fleeces by the Vortex Method Using the Pure L. lactis Powder without Prior Culturing (without Pre-Culturing)

L. lactis suspensions were prepared under sterile conditions in laminar air flow bench in MRS broth medium and saline at concentration of OD₆₀₀ of 1 McFarland by adding the L. lactis powder into 35 ml of MRS or saline in 50 ml centrifuge tubes and mixing. Each of the suspensions were distributed without pre-incubation in 3 centrifuge tubes 50 ml at 10 ml for each. Subsequently, the sterilized BNC fleeces were added to the tubes and loaded by the vortex method as previously described. The loaded BNC fleece was washed in saline, and transferred into 10 ml MRS in clear glass bottle of 30 ml. As control, a L. lactis culture was prepared by adding 5 μl from the L. lactis suspension at OD₆₀₀: 1 McFarland into 10 ml MRS in a clear glass bottle of 30 ml. The bottles were photographed (Canon PowerShot SX620HS) and were cultured in the incubator (Infors HT Multitron Standard) at 37° C. and 100 rpm for 24 h. After 24 h, the bottles were transferred to the laminar bench, were photographed (Canon PowerShot SX620HS) and the optical density (OD₆₀₀) was measured as previously described. 25 μl from each bottle were spread on the surface of a MRS agar plate using sterilized glass spreader and incubate (Heraeus 6000) at 37° C. for 48 h and the grown colonies on agar plate were photographed (Canon PowerShot SX620HS).

In the previous experiments the applied probiotics were always cultured in broth medium up to late log phase before use in the subsequence experiments and loading into the BNC. The experiment was designed to investigate the viability and survival rate of probiotic loaded into BNC fleece directly from powder without prior culturing in broth medium. The BNC fleeces were loaded by the vortex method using L. lactis suspensions prepared by adding the L. lactis powder to each; MRS broth medium and isotonic solution of saline NaCl 0.9% at OD₆₀₀: 1 McFarland.

The visual control of the L. lactis-loaded BNC fleeces after culturing demonstrated an obvious turbidity in the cultured bottles representing cell growth. The loaded L. lactis from both MRS and saline suspensions maintained a considerable viability and survivability and showed growth after incubation for 24 h, as confirmed by the measured OD₆₀₀. The loaded L. lactis from MRS and saline suspensions showed an OD₆₀₀ of 1.71±0.15 McFarland and 1.6±0.13 McFarland, respectively, after culturing under standard conditions. These data corresponded well with the observations from MRS-agar plates which showed a typical growth of L. lactis colonies.

Similar results were obtained when L. lactis powder was directly sprayed on the BNC fleeces applying compressed air.

### Example 4: Loading of B. subtilis Spore Powder in BNC Fleeces by Three Different Methods (Vortex, Injection and Spraying)

50 ml of B. subtilis spore suspension was prepared in sterile 0.9% NaCl at a concentration of OD₆₀₀ of 0.5 McFarland using the optical density spectrophotometer (Biophotometer) under sterile conditions in the laminar air flow bench. BNC fleeces were loaded with the B. subtilis spore suspension by the vortex method as described previously. Further BNC fleeces were loaded with the B. subtilis spore suspensions by the injection method at a concentration of OD₆₀₀ of 0.5 as described previously. Further BNC fleeces were loaded with the B. subtilis spore by the spray method as described previously.

All three different loading techniques were applicable for spore form of Bacillus, as an equal distribution of bacterial cells was confirmed in SEM micrographs. Re-culturing of the bacterial spores loaded by three different techniques showed viability both in medium and on agar plates.

### Example 5: Loading of Lactobacillus Spp. and Mixtures Thereof

The following strains were used: Lactobacillus fermentum, ID 51611, Lactobacillus rhamnosus, DSM 32609, Lactobacillus plantarum, DSM 32758.

The strains were cultured in MRS broth medium under aerobic standard conditions of 37° and 100 rpm shaking before they were suspended in Tris-magnesium buffer pH 7.4+50% glycerin and filled in cryo-tubes and stored at −80° C. until use. The aerobically cultured strains and several mixtures of them were then identified on MRS agar plates and microscopically characterized by SEM after fixing and drying by critical point dryer as described in previously.

Under sterile conditions in the laminar air flow bench (Heraeus HS 18/2), 4 sterilized 100 ml glass Erlenmeyer flasks were filled with 20 ml sterilized MRS broth bouillon medium at pH 6.2±0.2. 5 μl of each Lactobacillus strain was added into one flask, one flask was kept as MRS blank and the flasks were cultured at 37° C. and 100 rpm for 8 h in the orbital shaker incubator (Infors HT Multitron Standard). The flasks were transferred into the laminar bench (Heraeus HS 18/2) and the concentration of each strain was adjusted to OD₆₀₀ of 0.1 McFarland using the sterilized isotonic saline 0.9% NaCl and the optical density spectrophotometer (Biophotometer). 15 μl of the last adjusted bacterial suspension were added into 15 ml of MRS broth medium in 30 ml sterilized clear glass bottles and 3 bottles of each Lactobacillus strain were prepared. In another 15 ml MRS broth medium in 30 ml sterilized glass bottles, the different Lactobacillus strains were mixed at 5 μl of each, and 3 bottles for each mixture were prepared, as follow:
L. fermentum+L. rhamnosus
L. fermentum+L. plantarum
L. rhamnosus+L. plantarum
L. fermentum+L. rhamnosus+L. plantarum

The pH value of the prepared single and mixture cultures was measured before incubation and 5 ml of each bottle were transferred into 20 ml beaker glass and detect the pH value using the pH meter (Mettler Toledo 1140). All bottles were cultured at the same time in the orbital shaker incubator (Infors HT Multitron Standard) at 37° C. and 100 rpm for 8 h. After 8 h culturing, the bottles were transferred to the laminar bench (Heraeus HS 18/2) and the pH value was re-measured (Mettler Toledo 1140) of each culture as described above.

Loading the BNC fleeces with different mixtures of the Lactobacillus strains (L. fermentum, L. rhamnosus, L. plantarum) and evaluation of the pH changes of the cultured BNC: A culture of each Lactobacillus strain was prepared and the concentration of 90 ml of each was adjusted to OD₆₀₀ of 0.5 McFarland using saline as described above. In separate 50 ml tubes, the different Lactobacillus strain cultures were mixed with each other's as described above. 3 BNC fleeces were loaded with each Lactobacillus strain separately and with their mixture by the vortex method as described previously (and by spray loading as described previously) Under sterile conditions in the laminar air flow bench (Heraeus HS 18/2), each loaded BNC was added into 15 ml MRS broth medium in 30 ml sterilized clear glass bottle, and the pH value was measured before incubation (pH meter, Mettler Toledo 1140) as described above. All bottles were cultured in the orbital shaker incubator (Infors HT Multitron Standard) at 37° C. and 100 rpm for 8 h. After 8 h culturing, the bottles were transferred to the laminar bench (Heraeus HS 18/2) and the pH value was re-measured (Mettler Toledo 1140).

The Lactobacillus loaded BNC fleeces by vortex and spray method were fixed, dried and observed by SEM as described before.

The growth behavior of the Lactobacillus strains was investigated in an aerobic environment at the typical cultivation conditions of 37° C. and 100 rpm shaking in the selective MRS broth medium and on MRS-agar plate. All Lactobacillus strains, L. fermentum, L. rhamnosus and L. plantarum were grown in the broth medium demonstrated spherical colonies on the MRS-agar with various growth confluent. The colonies were white in color and showed a smooth surface. The SEM micrographs of the grown L. fermentum on MRS-agar showed the typical elongated Bacillus form at a size range of 1.5-3 μm and cell width of 0.5-0.7 μm, as single cells or grouped in pairs and short chains. Similarly, the L. rhamnosus displayed a bacillary form 1.0-2.7 μm long and 0.4-0.8 μm width, while the L. plantarum exhibited long rods with rounded ends at 2.5-5.5 μm long and 0.6-0.9 μm width. Moreover, different mixtures of the Lactobacillus strains were co-cultured in broth medium and the grown colonies were observed optically on the agar-MRS and microscopic by SEM.

The effect of the Lactobacillus growth on the pH value of the medium was investigated after culturing for 8 h at standard conditions. Particularly, all single strains and mixtures essentially reduced the pH value of the culture medium as presented in the table 1.

TABLE 1
pH values of the single and mixture cultures of Lactobacillus
strains before and after 8 h culturing

| | pH before | pH after | |
|---|---|---|---|
| Probiotics strain/mixture | culturing | culturing | P value |
| L. fermentum | 6.01 ± 0.01 | 4.57 ± 0.01 | <0.001 |
| L. rhamnosus | 6.02 ± 0.05 | 4.21 ± 0.09 | <0.001 |
| L. plantarum | 6.01 ± 0.04 | 4.35 ± 0.15 | 0.002 |
| L. fermentum + | 6.01 ± 0.02 | 4.35 + 0.07 | <0.001 |
| L. rhamnosus | | | |
| L. fermentum + | 6.0 ± 0.01 | 4.37 ± 0.03 | <0.001 |
| L. plantarum | | | |
| L. rhamnosus + | 6.01 ± 0.01 | 4.1 ± 0.08 | <0.001 |
| L. plantarum | | | |
| L. fermentum + | 6.0 ± 0.01 | 4.4 ± 0.1 | 0.001 |
| L. rhamnosus + | | | |
| L. plantarum | | | |

The pH values were significantly reduced (P<0.002) from 6.0±0.03 before culturing to 4.57±0.01, 4.21±0.09 and 4.35±0.15 after 8 h culturing for L. fermentum, L. rhamnosus and L. plantarum, respectively. Additionally, all mixtures of Lactobacillus strains also displayed a considerable reduction (P≤0.001) of pH values with 4.35±0.07, 4.37±0.03, 4.1±0.08 and 4.4±0.1 for L. fermentum+L. rhamnosus, L. fermentum+L. plantarum, L. rhamnosus+L. plantarum and L. fermentum+L. rhamnosus+L. plantarum, respectively. The reported reduction in the pH values of the mixture cultures was statistically significant P<0.05 comparing to the culture of each single strain, only the mixtures of L. rhamnosus+L. plantarum displayed no considerable difference in the pH values comparing to the single culture of each P>0.05.

Furthermore, the single Lactobacillus strains and several mixtures of them were loaded into BNC fleeces and observed by SEM, followed by culturing of the loaded BNC fleeces in MRS medium to determine the changes of the pH values. Accordingly, remarkable reduction of the pH values was also detected in all loaded BNC cultures P<0.001, table 2. The pH values of the media of the single Lactobacillus-loaded BNC before culturing were decreased from 6.0±0.01 to 4.59±0.02, 4.13±0.03 and 4.05±0.06 after 8 h culturing for L. fermentum-loaded BNC, L. rhamnosus-loaded BNC and L. plantarum-loaded BNC, respectively. Additionally, the mixtures of Lactobacillus-loaded BNC showed an obvious decrease of pH values demonstrated at 4.28±0.05, 4.38 0.01, 4.09±0.04 and 4.33±0.02 for L. fermentum+L. rhamnosus-loaded BNC, L. fermentum+L. plantarum-loaded BNC, L. rhamnosus+L. plantarum-loaded BNC and L. fermentum+L. rhamnosus+L. plantarum loaded BNC, respectively.

Moreover, loading of single or mixtures of Lactobacillus strains into BNC exhibited no considerable effect (P>0.05) on the pH value compared to the non-loaded cultured strains. Both, the loaded and non-loaded Lactobacillus strain demonstrated similar reduction of the pH values of the medium after 8 h culturing under standard aerobic conditions suggesting that the loading of probiotics in BNC fleece has no effect on their behavior.

TABLE 2
pH values of the single Lactobacillus-loaded BNC and mixtures
of Lactobacillus-loaded BNC before and after 8 h culturing

| single/mixture | pH before | pH after | |
|---|---|---|---|
| Lactobacillus-loaded BNC | culturing | culturing | P value |
| L. fermentum-loaded BNC | 6.02 ± 0.01 | 4.59 ± 0.02 | <0.001 |
| L. rhamnosus-loaded BNC | 6.0 ± 0.01 | 4.13 ± 0.03 | <0.001 |
| L. plantarum-loaded BNC | 6.01 ± 0.03 | 4.05 ± 0.06 | <0.001 |
| L. fermentum + | 6.0 ± 0.02 | 4.28 ± 0.05 | <0.001 |
| L. rhamnosus-loaded BNC | | | |
| L. fermentum + | 6.01 ± 0.01 | 4.38 ± 0.01 | <0.001 |
| L. plantarum-loaded BNC | | | |
| L. rhamnosus + | 6.01 ± 0.02 | 4.09 ± 0.04 | <0.001 |
| L. plantarum-loaded BNC | | | |
| L. fermentum + | 6.01 ± 0.01 | 4.33 ± 0.02 | <0.001 |
| L. rhamnosus + | | | |
| L.plantarum-loaded BNC | | | |

Similar results were obtained when Lactobacillus strains were loaded by spray technique as described before (see table 3).

TABLE 3
pH values of the single Lactobacillus-loaded BNC and mixtures
of Lactobacillus-loaded BNC before and after 8 h culturing

| | | | | P value |
|---|---|---|---|---|
| single/mixture | | | | Comparing |
| Lactobacillus-loaded BNC | pH before | pH after | | to vortex |
| by spray method | culturing | culturing | P value | method |
| L. fermentum-loaded BNC | 6.01 ± 0.01 | 4.38 ± 0.005 | <0.001 | <0.001 |
| L. rhamnosus-loaded BNC | 6.01 ± 0.01 | 3.90 ± 0.006 | <0.001 | 0.013 |
| L. plantarum-loaded BNC | 6.02 ± 0.01 | 3.94 ± 0.015 | <0.001 | 0.08 |
| L. fermentum + | 6.01 ± 0.02 | 4.24 ± 0.006 | <0.001 | 0.43 |
| L. rhamnosus-loaded BNC | | | | |
| L. fermentum + | 6.0 ± 0.01 | 4.19 ± 0.015 | <0.001 | <0.001 |
| L. plantarum-loaded BNC | | | | |
| L. rhamnosus + | 6.02 ± 0.02 | 3.94 ± 0.02 | <0.001 | <0.001 |
| L. plantarum-loaded BNC | | | | |
| L. fermentum + | 6.01 ± 0.01 | 4.17 ± 0.016 | <0.001 | <0.001 |
| L. rhamnosus + | | | | |
| L. plantarum-loaded BNC | | | | |

Similar results were obtained with L. delbrueckii DSM 32749 alone and in combination of L. delbrueckii, L. rhamnosus DSM 32609 and L. plantarum DSM 32758 by vortex and spray methods in its effect on the pH value and especially with regard to pathogen inhibition. As L. delbrückii shows weak growth under aerobic conditions and prefers anaerobic conditions, pre-culturing and pH-reduction-culturing was done under anaerobic conditions.

### Example 6: Preparation of Shelf-Stable Product by Spray and Vortex Technique (B. megaterium)

Preparation and Sterilization of BNC, Loading of Probiotics

In two 500 ml glass bottles under sterile conditions in laminar air flow bench (Heraeus HS 18/2), the BNC (mask, patch or other form) were immersed either in 50 ml broth medium of MRS and TSB or in an isotonic mixture of 0.9% NaCl+5% glucose. The BNCs were autoclaved in medium (Varioklav® 85T table-horizontal) at 121° C. and 1 bar or for 15 min. The BNC bottles were transferred into laminar air flow bench (Heraeus HS 18/2), and the BNC were removed from the medium, directly enfolded in aluminum compound foil and the foil was sealed by a welding seam (Famos F108). The medium- or NaCl/Glucose loaded BNCs were then subjected to E-Beam sterilization and sterile packed.

For loading 10 ml of the probiotic, bacterial suspensions were prepared in saline for both L. lactis and B. megaterium at a concentration OD₆₀₀ 0.5=10⁸ cells/ml. 5 ml of the probiotic suspension were sprayed on the BNC using a sterilized glass reagent sprayer. Loading was also performed by vortex method as described before.

The loaded BNC were freeze-dried using a freeze dryer (Epsilon 2-4 LSC, Martin Christ, Osterode, Germany) for 1-6 days, preferably for 3-5 days to a residual water content of between 3% and 14% (moisture analyzer; Ohaus MB45, Ohaus Corporation, USA). For ensuring flatness during drying process BNC fleeces were put between two foils. The residual water content was determined to be 13.92%±0.85%.

For long-term storage (at room temperature or 4° C. or temperature>30° C.) to assure re-swellablity (and stability), the freeze dried loaded BNC are packed in almost water-/humidity impermeable material, e.g. envelope the dried loaded mask in the mask pack envelope (Film composition PET/PE-/ALU/PE—12/15/9/50 μm) and closed thermally using the welding seam (Famos) or inner packaging foil and mask pack envelope.

Re-Culturing of Loaded BNC

The loaded BNC were transferred in broth medium (MRS for L. lactis-sprayed masks slices and TSB for B. megaterium-sprayed mask slices) in 30 ml sterilized glass bottle and re-cultured for 8 h at 37° C. and 100 rpm in an orbital shaker incubator (Infors HT Multitron Standard); blanks of MRS and TSB were incubated under the same conditions. After 8 h, the cultures were transferred from the incubator to the laminar air flow bench (Heraeus HS 18/2), the bottles were photographed, and after mixing 500 μl of each culture were collected in a sterilized 2 ml Eppendorf cup using a sterilized 1 ml pipette. The optical density OD600 nm of the collected samples was measured three times for each at a wavelength of 600 nm in comparison to the blank MRS or TSB medium using a UV cuvette and optical density spectrophotometer (Biophotometer). The slices cultures were spread on agar plates (MRS-agar for suspension of L. lactis-sprayed and vortex mask slices and TSB-agar for suspension of B. megaterium-sprayed and vortex mask slices) using the loop, and incubated plates at 37° C. for 24 h (Incubator Heraeus 6000), then agar plates were photographed.

Re-Swelling of Loaded BNC

One freeze-dried BNC mask was immersed in water (or alternatively in solution with further active ingredient) in glass beaker and re-swelled at room temperature for 10 min and the rolling ability of the re-swelled mask was evaluated. Another freeze-dried BNC mask was rolled, and the rolled BNC was immersed in water in 250 ml glass beaker for 10 min afterwards. A third freeze-dried BNC was rolled and transferred it in a 50 ml tube, then 20 ml water were added to the tube and kept for 10 min at room temperature.

The efficiency of probiotics loading on lip masks was investigated for both L. lactis and B. megaterium. The masks were autoclaved together with the corresponding broth medium followed by E-beam sterilization and spraying of the probiotic suspension on its surface. The probiotics-sprayed masks were then freeze-dried to hold the stability of probiotics and BNC material. Freeze-dried probiotics-loaded BNC was recultured in broth medium. The optical observation of the cultured bottles revealed a turbidity due to growth of the loaded probiotics. The freeze-dried L. lactis-loaded BNC demonstrated an OD₆₀₀ of 0.66±0.03 McFarland after culturing for 8 h. The reported OD₆₀₀ describe the quantity of L. lactis from 1 cm² of the mask surface. Additionally, the photographs of the spread suspension on MRS-agar plate exposed a typical white spherical colonies characteristic for L. lactis at confluent growth correlated to the measured OD₆₀₀. The results confirmed the survivility of the loaded L. lactis and its ability to proliferate after release from the mask.

The freeze-dried B. megaterium-loaded slices displayed a higher turbidity at OD₆₀₀ of 1.65±0.02 McFarland from 1 cm² of the mask surface. The grown colonies on TSB-agar demonstrated large smooth irregular colonies at white creamy in color identified for B. megaterium and ensured the stability and survivility of the loaded B. megaterium.

The re-swelling capacity of the isotonic mixture-loaded BNC mask was investigated in water at room temperature applying several approaches and forms. First, the freeze-dried loaded BNC mask was re-swelled in 100 ml water in glass beaker until the mask was re-swelled completely. In all approaches the BNC were re-swelled successfully within 10 min at room temperature.

Similar results were obtained for loading by vortexing.

### Example 7: Release of Loaded Probiotics

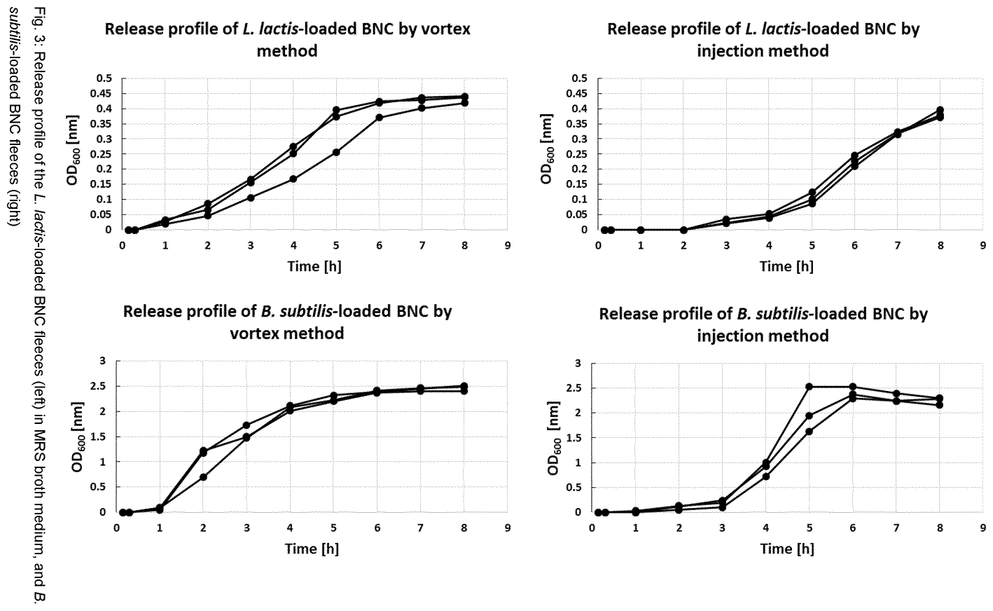

The release of the loaded probiotics from the BNC carrier is essential for the efficient biological activity at the site of effects. Therefore, the probiotic-loaded BNC fleeces prepared by vortex and injection methods were cultured in the corresponding broth medium to assess their release and proliferation profile at certain time points up to 48 h. The results indicate a constant increase of the probiotic counts in medium due to release and proliferation of the loaded probiotics as shown in FIG. 3.

FIG. 3 shows release profile of the L. lactis-loaded BNC fleeces (left) in MRS broth medium, and B. subtilis-loaded BNC fleeces (right) in TSB broth medium applying both vortex (top) and injection (bottom) loading methods. Results are given as mean of three independent measurements and presented up to 8 h for visualization purposes.

Both L. lactis and B. subtilis loaded by the vortex method were already detectable after 1 h in the broth medium and showed subsequently rapid proliferation up to 5 h followed by steady increase as illustrated in FIG. 3 (top). In contrast, the injection method offered the possibility for a more delayed release of loaded probiotics that were detected after 3 h (as shown in FIG. 3, bottom) followed by regular proliferation. Noticeably, the B. subtilis strain displayed a higher quantity reported at OD₆₀₀ of 2.5±0.1 McFarland and OD₆₀₀ of 2.4±0.2 McFarland after 8 h by vortex and injection method, respectively, in comparison to OD₆₀₀ of 0.44±0.2 McFarland and OD₆₀₀ of 0.38±0.2 McFarland of L. lactis by vortex and injection method, respectively. The results clearly demonstrate the efficiency of BNC as an appropriate carrier for the delivery of probiotics.

### Example 8: Re-Culturing of Probiotics from Freeze-Dried BNC

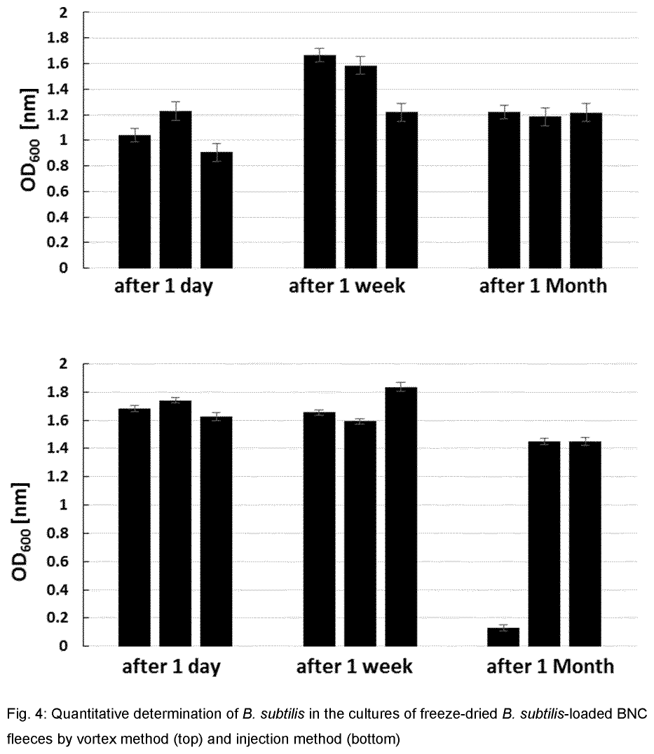

The stability of freeze-dried probiotics-loaded BNC fleeces (loaded by vortex method injection and spray method with L. lactis, B. subtilis and B. megaterium) were evaluated after different incubation times: 1 day, 1 week and 1 month, 3 months, 6 months by re-culturing. The freeze-dried control and probiotics-loaded BNC fleeces were incubated with broth medium (MRS for L. lactis-loaded BNC and TSB for B. subtilis-loaded BNC). The cultures were incubated at 37° C. and 100 rpm shaking for 8 h in orbital shaker incubator and the optical density OD₆₀₀ was determined in comparison to the control medium. FIG. 4 shows the quantitative determination of B. subtilis in the cultures of freeze-dried B. subtilis-loaded BNC fleeces by vortex method (top) and injection method (bottom) after 1-day, 1-week and 1-month storage period after culturing in TSB for 8 h. Results are given as mean±standard deviation of three independent measurements for each sample.

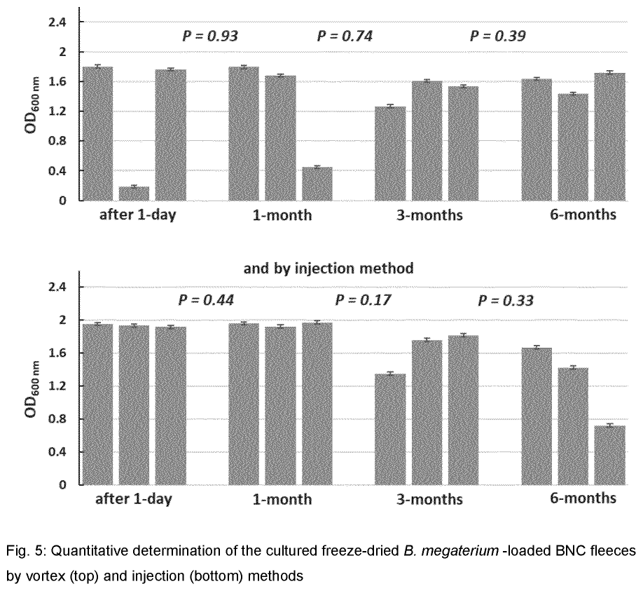

The results for B. megaterium for a storage period of 6 months are summarized in FIG. 5. FIG. 5 shows quantitative determination of the cultured freeze-dried B. megaterium-loaded BNC fleeces by vortex (top) and injection (bottom) methods over 6-months storage period at room temperature. Results are given as mean±standard deviation of three independent measurements.

The results for B. megaterium are summarized in table 4.

TABLE 4
The measured OD₆₀₀ ₙₘ of the cultured freeze-dried B. megaterium-loaded BNC by
vortex and injection methods over 6-months storage period at room temperature

| | OD_{600 nm} of the | OD_{600 nm} of the | | P value | P value |
|---|---|---|---|---|---|
| | B. megaterium - | B. megaterium - | P value | over time | over time |
| Storage | loaded BNC by | loaded BNC by | vortex- | interval by | interval by |
| period | vortex method* | injection method* | injection | vortex | injection |
| 1-day | 1.25 ± 0.91 | 1.93 ± 0.02 | P = 0.33 | — | — |
| 1-month | 1.31 ± 0.75 | 1.95 ± 0.03 | P = 0.27 | P = 0.93 | P = 0.44 |
| 3-months | 1.47 ± 0.18 | 1.64 ± 0.26 | P = 0.40 | P = 0.74 | P = 0.17 |
| 6-months | 1.6 ± 0.15 | 1.27 ± 0.49 | P = 0.37 | P = 0.39 | P = 0.33 |

*Results are given as mean ± standard deviation of three independent measurements

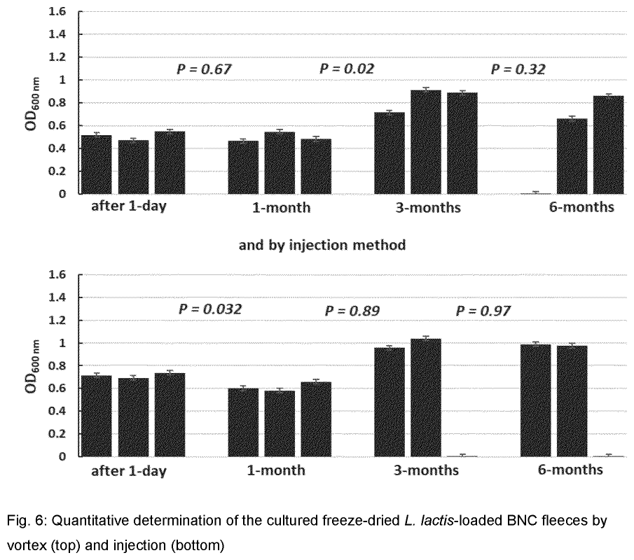

FIG. 6 shows the quantitative determination of the cultured freeze-dried L. lactis-loaded BNC fleeces by vortex (top) and injection (bottom) methods over 6-months storage period at room temperature. Results are given as mean±standard deviation of three independent measurements.

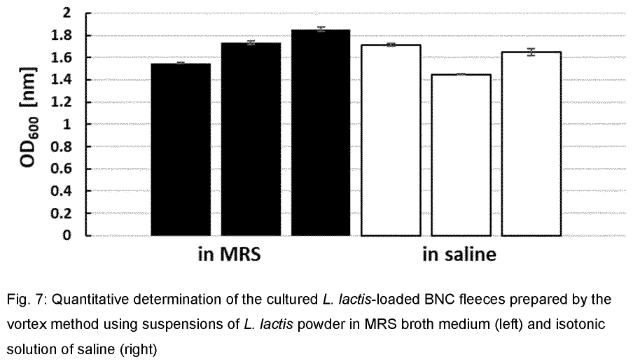

FIG. 7 shows the quantitative determination of the cultured L. lactis-loaded BNC fleeces prepared by the vortex method using suspensions of L. lactis powder in MRS broth medium and isotonic solution of saline without pre-culturing. Results are given as mean±standard deviation of three independent measurements for each sample. The results are summarized in table 5.

TABLE 5
The measured OD_{600 nm} of the cultured freeze-dried L. lactis-loaded BNC by
vortex and injection methods over 6-months storage period at room temperature

| | OD_{600 nm} of the | OD_{600 nm} of the | | P value | P value |
|---|---|---|---|---|---|
| | L. lactis-loaded | L. lactis-loaded | P value | over time | over time |
| Storage | BNC by vortex | BNC by injection | vortex- | interval by | interval by |
| period | method* | method* | injection | vortex | injection |
| 1-day | 0.51 ± 0.39 | 0.72 ± 0.22 | P = 0.003 | — | — |
| 1-month | 0.46 ± 0.03 | 0.61 ± 0.4 | P = 0.027 | P = 0.67 | P = 0.032 |
| 3-months | 0.84 ± 0.11 | 0.66 ± 0.58 | P = 0.65 | P = 0.02 | P = 0.89 |
| 6-months | 0.51 ± 0.45 | 0.65 ± 0.57 | P = 0.74 | P = 0.32 | P = 0.97 |

*Results are given as mean ± standard deviation of three independent measurements

Further, the loading capacity of the probiotics L. lactis and B. subtilis in modified BNC fleece was compared to standard BNC fleece and evaluated.

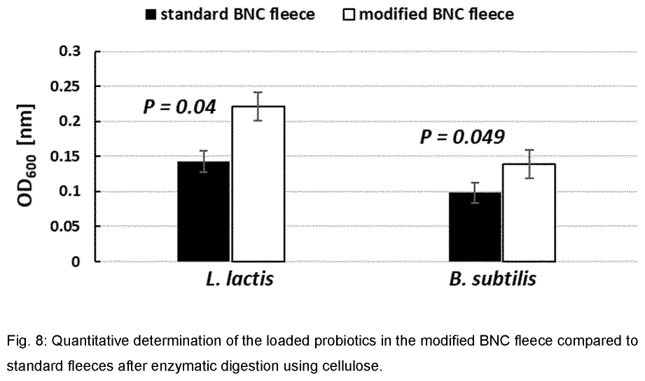

FIG. 8 shows the quantitative determination of the loaded probiotics in the modified BNC fleece compared to standard fleeces after enzymatic digestion using cellulose. Results are given as mean±standard deviation of three independent measurements for each sample.

Similar results were obtained for loading by spraying.

### Example 9: Production Process and Bacterial Cellulose Based Product Containing Probiotics/Synbiotics for Topical Applications

For topical applications potential products include: thin masks, patches, 3D BNC products: face masks and lip masks, and sanitary products, such as panty liner, tampons and sanitary towels.

Pre-synthesized BNC (as masks, patches or other 3D products, e.g. tamponades) were prepared by loading of medium or NaCl/glucose solution, also in combination with the loading of nutrients and technical aids. The BNC (e.g. mask) are immersed in glass bottles under sterile conditions in laminar air flow bench (Heraeus HS 18/2, in 50 ml medium, e.g. MRS and TSB). Alternatively, the BNC masks are immersed in an isotonic mixture of 0.9% NaCl+5% glucose, and the loaded masks were freeze-dried and sterilized as described in Example 6. The prepared BNC were then loaded with probiotics and active ingredient nutrients using different techniques:

Loading of BNC Masks by Spraying:

10 ml probiotic suspension in saline of probiotic were prepared (e.g. L. lactis and B. megaterium) concentration OD₆₀₀ of 0.5. 5 ml of the probiotic suspension was homogenously sprayed on the BNC (e.g. masks) using the sterilized glass reagent sprayer.

Loading of BNC Masks by Vortex:

BNC fleeces were added to the probiotic suspension in 50 ml tubes, 3 tubes were prepared for each probiotic strain and BNC fleeces were added into sterilized medium or saline. The tubes were vortexed (Vortexer Genie 2) using the multi tube holder (SI-V506 vertical 50 ml tube holder) at vortex strength 10.5 in room temperature for 10 min. The loading suspension was removed, and the BNC were washed in 10 ml saline under vortex for 10 sec.

Drying of the Loaded BNC Masks

The probiotics-loaded BNC masks were dried using the freeze dryer (Epsilon 2-4 Isc Christ). Freeze drying together assures 3D structure for re-swelling capacity. Masks/patches were put between a bottom and a top foil during freeze drying to ensure for optimal flatness of dried BNC fleeces.

The loaded BNC were freeze-dried using a freeze dryer (Epsilon 2-4 LSC, Martin Christ, Osterode, Germany) for 1-6 days, preferably for 3-5 days to a residual water content of between 3% and 14% (moisture analyzer; Ohaus MB45, Ohaus Corporation, USA). When BNC does not reach the defined residual water content of max. 14% during drying, re-swelling capacity is negatively influenced and stability can be shortened.

Packaging

For long-term storage (at room temperature or 4° C. or temperature>30° C.) to assure re-swellablity (and stability) the freeze-dried loaded BNC are packed in almost water-/humidity impermeable material. The packaging material for the packaging foil is an aluminum compound foil consisting of polyethylene terephthalate (PET), aluminum (Al) and polyethylene (PE), e.g. envelope the dried loaded mask in the mask pack envelope (e.g. PET/PE-ws/ALU/PE—12/15/9/50 μm) and closed thermally using the welding seam (Famos) or inner packaging foil (PET, 50 μm) and mask pack envelope. The packaging material for the packaging foil is an aluminum compound foil consisting of polyethylene terephthalate (PET), aluminum (Al) and polyethylene (PE), (Tesseraux, Buerstadt, Germany or Gruber Folien, Straubing. Germany).

Use of the Product

Before using the BNC mask, the BNC mask needs to be removed from packaging and re-swelled e.g. with water before use to soften the BNC material for use and re-activate probiotics or re-swelled with liquid containing active ingredients (in case of anti-inflammatory mask) to soften BNC mask and re-activate probiotics and activate probiotics.

### Example 10: Anti-Inflammatory Mask Product: BNC Loaded with B. megaterium (by Spray Technique and Vortexing) for Anti-Inflammation Topical Use

Materials:

As strains for anti-inflammatory topical application Bacillus megaterium strains were used, especially B. megaterium DSM 32963 & DSM 33300 & DSM 33336. Moreover, the BNC were loaded with an anti-inflammatory omega-3 lysine salt (AvailOm®), which contains around 32 weight-% of L-lysine and around 65 weight-% of polyunsaturated fatty acids, mainly eicosapentaenoic acid (EPA), docosahexaenoic acid (DHA).

The BNC masks were synthesized, cleaned and sterilized before they were loaded with the isotonic mixture of 0.9% NaCl and 5% glucose. Under sterile conditions in the laminar air flow bench (Heraeus HS 18/2) 25 μl of the B. megaterium cryo-stock suspension were add into 150 ml TSB broth medium in sterilized 250 ml glass Erlenmeyer flask, the flask was closed with a cork stopper and cultured for 8 h at 37° C. and 100 rpm in the orbital shaker incubator (Infors HT Multitron Standard). After 8 h, the culture was transferred to the laminar air flow bench (Heraeus HS 18/2), the culture was distributed in 3×50 ml centrifuge tube and centrifuged at room temperature and 4000 rpm for 20 min using the tube centrifuge (Eppendorf centrifuge 5804R). The supernatant was removed, and the precipitate was resuspended in the previously warmed (37° C.) sterilized isotonic saline (0.9% NaCl). The optical density of the B. megaterium suspension was adjusted in saline into OD₆₀₀ of 0.5 using the optical density spectrophotometer (Biophotometer).

Under sterile conditions in the laminar air flow bench (Heraeus HS 18/2) und by using a plastic tweezer, each mask was transferred on the inner packaging foil (PET, 50 μm). The mask was loaded with the B. megaterium suspension in saline by spraying 5 ml homogenously on each surface using the sterilized glass reagent sprayer. The loaded mask was covered with the second inner packaging foil (PET, 50 μm), then freeze-dried for 5 days in the freeze dryer (Sublimator 3×4×5, Zirbus technology GmbH, Germany) until residual water content of max. 14%. After freeze-drying, the mask was enfolded in the mask pack envelope and closed thermally using the welding seam (Famos) and the packaged product was stored. Stability testing were performed for storing at 4° C., RT, 30° C. and 40° C.

To analyze the re-swelling capacity and stability of the freeze-dried isotonic mixture—and B. megaterium-loaded lip mask after 6-months storage period at different temperatures the mask was loaded with the isotonic mixture of 0.9% NaCl+5% glucose and with the probiotic B. megaterium then freeze dried and stored enveloped in aluminum compound foil at 4° C. as described before. The re-swelling capacity and the B. megaterium stability was evaluated as described in example 6.

For evaluation of the re-swelling capacity of the BNC lip mask and the survivility of the loaded B. megaterium after 2-months storage period at 30° C. and 40° C. the masks were re-swelled in 20 ml water at room temperature for 10 min. Under sterile conditions in the laminar air flow bench (Heraeus HS 18/2), masks were opened and 3 slices (1×1 cm) from each mask were cultured in 10 ml TSB broth medium for 8 h at 37° C. and 100 rpm in the orbital shaker incubator (Infors HT Multitron Standard) followed by measuring the optical density of the obtained cultures for quantitative determination and spreading on TSB-agar plate for qualitative observations.

The re-swelling capacity of the isotonic mixture-und B. megaterium-loaded BNC mask was investigated after freeze-drying and storage at 4° C. for 6 months or at RT for 5 months. Accordingly, the mask slices maintained the large re-swelling ability and showed a remarkable increase of the volume. The mask slices rapidly returned the initial shape within 7-10 min and showed a significant weight increase P=0.001 from 0.019±0.001 g to 0.27±0.019 g and confirmed the re-swelling capacity of the prepared BNC masks during storage at 4° C. for the considered time.

Table 6 summarizes the re-swelling capacity of the freeze-dried isotonic mixture- and B. megaterium-loaded lip mask over 6-months storage period at 4° C. The dried slices maintained the re-swelling capacity and exhibited significant weight increase P<0.05 within 7-10 min in water at room temperature after storage at 4° C. up to the mentioned storage periods. The observed variability in the detected weight increase between time intervals were all statistically non-significant P>0.05. The stability and viability of the loaded B. megaterium in the BNC mask was also evaluated after 6-months storage period. The cultured slices of the loaded BNC mask showed notable turbidity under the standard culturing conditions and demonstrated remarkable growth.

Table 7 summarizes the quantitative determination of the cultured freeze-dried isotonic mixture—and B. megaterium-loaded lip mask slices over 6-months storage period at 4° C. The cultured slices exhibited also remarkable viability and activity of the loaded B. megaterium and reported a considerable grown quantity at OD₆₀₀ of 1.48±0.24 McFarland. A significant increase at P=0.035 in the measured grown quantity were detected after 3-months storage period, this increase could be related to the increased loaded number of the B. megaterium or to non-homogeneous spray of the probiotic's suspension on the surfaces of the BNC masks.

TABLE 6
Weight of the dried and re-swelled slices from the freeze-
dried stored isotonic mixture- and B. megaterium-loaded
lip mask after storage at 4° C. over 6 months

| | | Weight of | | |
|---|---|---|---|---|
| | Weight of | re-swelled slice | P value | P value |
| Storage | stored dried | [g] after 10 min in | dried- | over time |
| period | slice [g]* | water* | reswelled | interval |
| 1-day | 0.014 ± 0.001 | 0.212 ± 0.023 | P = 0.004 | — |
| 1-month | 0.014 ± 0.002 | 0.208 ± 0.031 | P = 0.007 | 0.16 |
| 3-months | 0.015 ± 0.001 | 0.23 ± 0.017 | P = 0.002 | 0.1 |
| 6-months | 0.019 ± 0.001 | 0.27 ± 0.019 | P = 0.001 | 0.23 |

*Results are given as mean ± standard deviation of three independent measurements

Re-swellability and stability by testing viability after re-culturing was shown. When masks were not dry enough at packaging time fungus growth could be detected. This was not the case, when masks were completely dried to a maximum residual water content of 14% after freeze drying procedure.

TABLE 7
The measured OD600 nm of the cultured freeze-dried isotonic
mixture- and B. megaterium-loaded lip mask slices in
TSB broth medium after storage at 4° C. over 6 months

| storage period | 1-day | 1-month | 3-months | 6-months |
|---|---|---|---|---|
| * OD_{600 nm} | 1.32 ± 0.06 | 1.35 ± 0.02 | 1.85 ± 0.83 | 1.41 ± 0.035 |
| P value over | — | 0.44 | 0.035 | 0.08 |
| time interval | | | | (1-6 month) |

* Results are given as mean ± standard deviation of three independent measurements

Similar results with regard to re-swellability and viability were obtained for storage at room temperature, 30° C. and 40° C. after proper freeze-drying and packaging. Packing in foil was suitable but better results were obtained with 2 inner foils (as described before) before packaging into sealed outer foil.

Samples from isotonic mixture and B. megaterium-loaded BNC lip mask for measurement of the specialized pro-resolving mediators (SPM) and their precursors were prepared. Two BNC lip masks were loaded with the isotonic mixture- and B. megaterium, then freeze-dried and re-swelled and the first freeze-dried BNC mask was loaded using (1) 0.01% liposomal AvailOm® aqueous suspension and (2) the second mask with 0.01% powder AvailOm® aqueous solution. Then slices from the re-swelled masks were cultured in TSB broth medium and on TSB-agar plate at the standard conditions. Alternatively, two BNC lip masks were firstly loaded with the isotonic mixture. Afterwards, the B. megaterium was added at a concentration of OD₆₀₀ of 0.5 McFarland to each; (1) 0.01% liposomal AvailOm® aqueous suspension, and (2) to 0.01% powder AvailOm® aqueous solution. Afterwards, B. megaterium-AvailOm® mixtures were sprayed on one mask, then freeze-dried and re-swelled in water. The slices from the re-swelled masks were cultured in TSB broth medium and on TSB-agar plate as described above.

The slices from non-loaded BNC masks were cultured in broth TSB and on TSB-agar as control. The cultured slices in both, broth TSB medium and on TSB-agar were then prepared for SPM measurements and their precursors). The broth medium is diluted in methanol at 2:1 V in 50 ml tubes. The agar with the cultured slices (2×2 cm) is transferred into another 50 ml tube and 8 ml methanol are added, then both the broth medium and agar samples are cooled at −20° C. for 60 min and centrifuged at 4500 rpm for 10 min. Finally, the supernatant is collected in separate tubes for quantitative and qualitative determination of the SPM compared to the controls of cultured non-loaded BNC mask slices prepared using the same procedure.

The production of specialized pro-resolving mediators (SPM) and their precursors from the loaded B. megaterium-AvailOm® mixture on BNC lip mask was investigated in broth medium and on agar plate. Both AvailOm® forms; liposomal and powder were loaded with the B. megaterium on the BNC mask applying two sequences pathways. In the first way (A), the liposomal AvailOm® suspension or the powder AvailOm® solution was used to re-swell the freeze-dried B. megaterium-loaded BNC mask. While the AvailOm® suspension/solution, in the second way (B), was mixed with the B. megaterium and sprayed on the BNC mask before the freeze drying, followed by re-swelling by water. Subsequently, slices from the re-swelled B. megaterium- and AvailOm®-loaded BNC lip mask were cultured in TSB broth medium and on TSB-agar plate to determine the production of SPM comparing to controls of non-loaded BNC mask slices in TSB medium and on TSB-agar. Accordingly, several lipid mediators generated by lipoxygenases, cytosolic phospholipase A2, cyclooxygenase 1 or 2 were measured by ultraperformance liquid chromatography mass spectrometry UPLC-MS.

SPMs are known for its natural inflammation-resolving activities. Thus, the above described resulting anti-inflammatory mask/path is for topical anti-inflammatory treatment on skin or mucous membranes. Most prominently the following SPMs were produced:

17-HDHA 17-hydroxy Docosahexaenoic Acid, 14-HDHA 14-hydroxy Docosahexaenoic Acid, 13-HDHA 13-hydroxy Docosahexaenoic Acid, 7-HDHA 7-hydroxy Docosahexaenoic Acid, 4-HDHA 4-hydroxy Docosahexaenoic Acid, 15-HEPE 15-hydroxy Eicosapentaenoic acid, 12-HEPE 12-hydroxy Eicosapentaenoic acid, 11-HEPE 11-hydroxy Eicosapentaenoic acid, 5-HEPE 5-hydroxy Eicosapentaenoic acid, 15-HETE 15-Hydroxyeicosatetraenoic acid, 12-HETE 12-Hydroxyeicosatetraenoic acid, 11-HETE 11-Hydroxyeicosatetraenoic acid, 8-HETE 8-Hydroxyeicosatetraenoic acid, 5-HETE 5-Hydroxyeicosatetraenoic acid, AA Arachidonic acid, EPA Eicosapentanoic acid, DHA Docosahexanoic acid, PD1 Protectin D1, AT-PD1 aspirin triggert-Protectin D1, PDX Protectin DX, RvD5 Resolvin D5, MaR1 Maresin 1, MaR2 Maresin 2, t-LTB4 trans-Leukotrien B4, LTB4 Leukotrien B4, 20-OH-LTB4 20-Hydroxy-Leucotrien B4, PGE2 Prostaglandin E2, PGF2a Prostaglandin F2alpha, TXB2 Tromboxan B2, LXA4 Lipoxin A4, AT-LXA4 aspirin triggert-Lipoxin A4, LXA5 Lipoxin A5, RvD1 Resolvin D1, RvD4 Resolvin D4

### Example 11: BNC Patch/Mask with Bacillus subtilis for Staphylococcus aureus Inhibition

Loading was performed with three different methods (vortex, spray and injection as described previously.

For supernatant preparation 35 ml of the last cultured bacterial suspension of each B. megaterium DSM 32963 and B. subtilis DSM 33561 were centrifuged in 50 ml centrifuge tube at 4500 rpm at 4° C. for 30 min using the tube centrifuge (Eppendorf centrifuge 5804R). The supernatant was collected in 50 ml syringe and filtrate it into other 50 ml centrifuge tube using syringe filter 0.2 μm.

Under sterilized conditions in laminar air flow bench (Heraeus HS 18/2), S. aureus were added at concentration OD₆₀₀ of 0.1 McFarland into 10 ml of the probiotic free supernatant of both B. megaterium and B. subtilis-free supernatant in 30 ml sterilized glass bottle. 5 ml of S. aureus were added at concentration OD600 0.1 McFarland into 5 ml of B. megaterium or B. subtilis suspension at concentration OD600 0.1 McFarland in 30 ml sterilized glass bottle. The positive control was prepared by adding gentamicin at concentration 300 μg/ml into TSB medium, then adding the S. aureus at concentration OD600 0.1 McFarland. The bottles were incubated in the orbital shaker incubator (Infors HT Multitron Standard) at 37° C. and 100 rpm for 18 h. After 18 h the bottle was transferred into laminar air flow bench (Heraeus HS 18/2) and photographed. 5 μl of each bottle was spread on TSB-agar using the loop and incubate the agar plate at 37° C. for 24 h (Incubator Heraeus 6000) and the agar plate was photographed

For the agar diffusion test the OD₆₀₀ of B. megaterium and B. subtilis was adjusted to 0.1 McFarland using sterilized saline NaCl 0.9%. The OD₆₀₀ of S. aureus was adjusted to 0.5 McFarland using sterilized saline NaCl 0.9%. 20 μl of S. aureus was spread on the surface of Mueller-Hinton agar plate by a sterilized glass spreader. The wells are melted on the agar plate using the back side of a 1 ml pipette tip. A small volume of Mueller-Hinton agar was melted in boiling water bath, then 100 μl of it was used to close the bottom of each created well. After solidification of the agar in the bottom of the wells, the wells were filled with 100 μl of: Negative control, sterilized saline NaCl 0.9%, Positive control, gentamicin 300 μg/ml, B. megaterium or B. subtilis-free supernatant, B. megaterium or B. subtilis suspension. The agar plates were incubated at 37° C. for 24 h (Incubator Heraeus 6000) and photographed afterwards and the inhibition zones were determined.

The evaluation of the antibacterial activity of B. subtilis and B. megaterium loaded-BNC against gram-positive S. aureus was determined by an agar diffusion test. Therefore, the bacterial suspensions of B. subtilis and S. aureus were prepared in TSB broth medium as described before. The B. subtilis free supernatant was prepared and the BNC fleeces were loaded with B. subtilis by vortex method. 3 BNC fleeces were loaded using B. subtilis suspension in TSB medium, and 3 BNC fleeces were loaded using B. subtilis suspension in saline. Further 3 BNC fleeces were loaded using the B. subtilis free-supernatant, 3 BNC fleeces were loaded with gentamycin as positive control, and 3 BNC fleeces with isotonic saline as negative control. The OD₆₀₀ of S. aureus was adjusted to 0.5 McFarland using sterilized saline NaCl 0.9% and the optical density spectrophotometer (Biophotometer) was determined. 20 μl of S. aureus was spread on the surface of Mueller-Hinton agar plate by a sterilized glass spreader. The last control and loaded BNC fleeces were added onto surface of the Mueller Hinton agar: 1. Negative control: saline-loaded BNC 2. Positive control: gentamicin-loaded BNC 3. B. subtilis-loaded BNC in TSB medium 4. B. subtilis-loaded BNC in saline 5. B. subtilis-free supernatant-loaded BNC. The agar plates were incubated at 37° C. for 24 h (Incubator Heraeus 6000), photographed afterwards and the inhibition zones were determined.

The inhibition activity of each probiotic B. megaterium and B. subtilis against gram-positive S. aureus was tested before loading onto BNC. Only B. subtilis was effective in inhibiting S. aureus. The S. aureus was incubated with each; the probiotics suspension and the probiotics-free supernatant prepared by culturing over 24 h. The obtained results from co-culturing test showed a turbidity in the prepared cultures. To classify the grown strain and to detect the inhibition effect, the turbid suspensions were spread on agar plate along with the control of each probiotics and S. aureus strain. The photographs of the agar plates indicated no inhibition effect of B. megaterium on S. aureus. Neither the B. megaterium suspension nor the B. megaterium-free supernatant displayed any inhibition effect on S. aureus. Whereas, a remarkable inhibition of B. subtilis DSM 33561 was detected against S. aureus. The B. subtilis colonies were only observed on the surface of the tested plates without any detected growth of S. aureus colonies on both B. subtilis suspension and B. subtilis-free supernatant plates. These results were further reinforced by agar well diffusion test. B. megaterium plates showed an inhibition zone on gentamicin well, while no inhibition zone was detected on B. megaterium suspension or the B. megaterium-free supernatant. The B. subtilis suspension displayed an inhibition zone of 0.5±0.1 mm in radius associated with growth of B. subtilis colonies on the well. However, contrast to the results of the co-culturing test, the B. subtilis-free supernatant well demonstrated no inhibition zone, which could be related maybe to the low concentration of the effective molecules in the used volume of the supernatant. For further B. subtilis strains, the obtained results from the co-culturing test were further reinforced by the standard agar well diffusion test. A remarkable inhibition zone around both the B. subtilis-free supernatant and B. subtilis cells-containing wells could be detected, associated with considerable growth around the well edge.

After loading of the bacterial cultures onto BNC, the antibacterial activity of B. subtilis against gram-positive S. aureus was shown by two standard tests; co-culturing test and agar well diffusion test. The probiotics (B. subtilis, B. megaterium) were loaded into BNC by vortex, spray and injection method using TSB broth medium and isotonic saline as loading solutions. Antibacterial activity of the loaded B. subtilis in the BNC fleeces against S. aureus manifested by a marked inhibition zone around the loaded BNC fleeces using both TSB broth medium and saline with vortex (3-4 mm inhibition zone) and spray method (5 mm inhibition zone), but not by injection, and with neither loading method for B. megaterium. Simultaneously, B. subtilis colonies were grown near the BNC. An inhibition zone was also detected around the B. subtilis-free supernatant-loaded BNC by vortex (1-3) and spray method (>2 mm). Surprisingly, for inhibition by B. subtilis on BNC, also the cell-free extract was effective in contrary to the pure on loaded cell-free extract of B. subtilis DSM 33561. when loading was done by vortex or spraying. The results are summarized in table 8.

TABLE 8
Summary of inhibition effects of B. subtilis and B. megaterium
cells and cell free supernatant (by detection of inhibition zone >
2 mm) on S. aureus by diffusion test with and without BNC

| | | on BNC - | on BNC - | on BNC - |
|---|---|---|---|---|
| | | vortex | spray | injection |
| | Without BNC | technique | technique | technique |
| Gentamycin - | + | + | + | + |
| positive control | | | | |
| Saline - negative | − | − | − | − |
| control | | | | |
| B. subtilis with | + | + | + | − |
| medium | | | | |
| B. subtilis cells | + | + | + | − |
| with saline | | | | |
| B. subtilis cell- | − | + | + | − |
| free supernatant | | | | |
| B. megaterium | − | − | − | − |
| cells | | | | |
| B. megaterium | − | − | − | − |
| cell-free | | | | |
| supernatant | | | | |

Similar inhibitory results were detected for further B. subtilis strains namely B. subtilis DSM 33353 and DSM 33298.

### Example 12: Probiotics on BNC for Feminine/Vaginal Health Products with Lactobacillus Spp. or Lactococcus Spp.

Probiotics single or mixture are loaded on BNC (thin layer or 3D structure) e.g. as layer in panty liner, sanitary towels, or rolled as tampons or as a three dimensional structure as tampons or tamponage, taking into account the re-swelling capacity of BNC and the carrier/loading capacity for probiotics. Loaded probiotics help to maintain vaginal milieu by pH reduction, H₂O₂ production or urogenital pathogen inhibition. For those applications, the following strains were used: Lactobacillus rhamnosus, DSM 32609, Lactobacillus fermentum Lactobacillus plantarum, DSM 32758, Lactobacillus delbrueckii susp. bulgaricus DSM32749.

Evaluation of the Re-Swelling Capacity of Flat and Rolled BNC in Water

For a product in the form of a tampon or layer for panty liner, 4 BNC fleeces (10×10 cm) were immersed in 400 ml isotonic mixture of 0.9% NaCl+5% glucose, then autoclaved and freeze-dried as described before. The freeze-dried BNC fleece was immersed in 100 ml water in 250 ml glass beaker, re-swelled at room temperature for 10 min, then the rolling ability of the re-swelled mask was evaluated. A second freeze-dried BNC mask was rolled and immersed in 100 ml water in 250 ml glass beaker for 10 min. A third freeze-dried BNC fleece was rolled and transferred it in a 50 ml tube, then 20 ml water were added to the tube and kept for 10 min at room temperature. A fourth freeze-dried BNC fleece was rolled, transferred it in a 50 ml tube, and the tube was set overturned in petri dish, then 20 ml water was added to the petri dish and kept 10 min at room temperature.

The re-swelling capacity of the isotonic mixture-loaded BNC fleece was investigated in water at room temperature applying several approaches and forms. First, the freeze-dried loaded BNC mask was re-swelled in 100 ml water in glass beaker, the mask was completely re-swelled after 10 min and showed flexibility and ability for rolling after re-swelling.

Secondly, the freeze-dried loaded mask was rolled before the re-swelling was completed in water in a glass beaker for 10 min at room temperature. The mask was rolled off during the re-swelling process and returned to the initial flat form after 10 min in water. Moreover, the third freeze-dried loaded BNC fleece was rolled and re-swelled in water using a tube similar to a vaginal cavity. The fleece was completely re-swelled and filled the whole tube, while the placement of the fleece in a tube overturned in a petri dish filled with water provided slower re-swelling only starting at the bottom part of the fleece which is in contact with fluid without rolling off. Preferable for application is therefore a short pre-wetting of flat or rolled BNC fleeces to enable for use and easy re-swelling.

Loading of BNC with Lactobacillus Strains

Loading of Lactobacillus spp. and mixtures thereof and the pH reduction is described in example 5.

Similar results for distribution of bacterial cells on the BNC non-woven were obtained when Lactobacillus strains where loaded by spray technique as described before.

For L. delbrueckii subsp. bulgaricus DSM 32749 suitability to perform for feminine health especially in combination with L. plantarum DSM 32758 or a three-strain combination also comprising L. rhamnosus DSM 32609 was also shown. In this case protocol was adapted to account for its preferred anaerobic cultivation. Cultivation was performed in MRS medium under anaerobic conditions. All strains were also able to grow in simulation of vaginal fluid (MSVF).

Furthermore, additional strains of Lactobacillus spp. and/or Lactococcus species could be used alone or in combination for the products, especially when a potential for feminine health was shown (e.g. by pH reduction, H₂O₂ production or pathogen inhibition e.g. uropathogenic E. coli).

In a preferred embodiment the strains are selected from DSM 33370 L. plantarum LN5, DSM 33377 L. brevis LN32, DSM 33368 L. plantarum S3, DSM 33369 L. plantarum S11, DSM 33376 L. paracasei S20, DSM 33375 L. paracasei S23, DSM 33374 L. reuteri F12, DSM 33367 L. plantarum F8, DSM 33366 L. plantarum S4, DSM 33364 L. plantarum S28, DSM 33363 L. plantarum S27, DSM 33373 L. paracasei S18a, DSM 33365 L. plantarum S18b, DSM 33362 L. plantarum S13, DSM 32767 Lactococcus lactis sups. lactis, L. fermentum DSM 32750

### Example 13: BNC Mask/Patch with Propionibacterium acnes/Cutibacterium Acnes for Anti-Acne Masks

Glucose/NaCl-prepared BNC non-woven (as patch or mask) was loaded with Cutibacterium acnes by vortexing and spray loading technique and freeze dried and packed for storing as described previously in example 9. Re-swelling and stability testings showed suitability of the described process also for this product application. This product example has the focus of topical anti-acne application by beneficial influence of Cutibacterium acnes in pathogenic acne microflora after application of mask/patch.

### Example 14: BNC Mask/Patch with S. epidermidis for Re-Balancing/Influencing Skin Microbiome

Glucose/NaCl-prepared BNC non-woven (as patch or mask) was loaded with Staphylococcus epidermidis by vortexing and spray loading technique and freeze dried subsequently and packed for storing as described previously in example 9. Re-swelling and stability testings showed suitability of the described process also for this product application. This product example has the focus of topical re-balancing of skin microflora by beneficial influence of S. epidermidis on topical microbiome composition after application of mask/patch.

1. A method for loading microorganisms or parts thereof on and/or in a pre-synthesized multiphase biomaterial comprising nanocellulose, the method comprising:
synthesizing a bacterially synthesized nanocellulose (BNC) multiphase biomaterial,
resuspending the microorganisms in a buffer or a culture medium, and
loading the microorganisms into and/or onto the BNC material by either:
mixing the multiphase biomaterial with the microorganisms at 300 rpm or more at a temperature of 37° C. or less, or
injecting the microorganisms into the multiphase biomaterial and incubating at a temperature of 37° C. or less, or
incubating the multiphase biomaterial in the buffer or culture medium with resuspended microorganisms at a temperature of 37° C. or less for 60 min or less.

2. The method of claim 1, wherein the microorganisms are sprayed onto the multiphase biomaterial.

3. The method of claim 1, wherein the microorganisms are loaded as vegetative cells or in a dormant form, or as a cell-extract.

4. The method of claim 1, wherein the microorganisms are wet or dry and/or pre-cultured or not pre-cultured.

5. The method of claim 1, wherein the multiphase biomaterial is wet or dried or partially dried or re-swelled in buffer.

6. The method of claim 1, wherein the nanocellulose is derived from a plant, algae, or a microorganism.

7. The method of claim 1, wherein the bacterially synthesized nanocellulose (BNC) comprises a layered structure.

8. The method of claim 1, wherein the bacterially synthesized nanocellulose (BNC) is a BNC non-woven with an average thickness of at least 0.5 mm.

9. The method of claim 1, wherein at least two different bacterial cellulose networks are designed as a combined homogenous phase system or as a layered phase system comprising at least one combined homogenous phase and at least one single phase.

10. The method of claim 1, wherein further substances are added during bacterial synthesis of BNC to control the resulting pore/mesh sizes.

11. The method of claim 1, further comprising:
incubating the loaded multiphase biomaterial with a moisture binder for drying, wherein the moisture binder is an osmotically and/or hygroscopically effective solution.

12. The method of claim 1, wherein the microorganism is a probiotic bacterial or yeast strain is at least one selected from the group consisting of Bifidobacterium, Carnobacterium, Corynebacterium, Cutibacterium, Lactobacillus, Lactococcus, Leuconostoc, Microbacterium, Oenococcus, Pasteuria, Pediococcus, Propionibacterium, Streptococcus, Bacillus, Geobacillus, Gluconobacter, Xanthonomas, Candida, Debaryomyces, Hanseniaspora, Kluyveromyces, Komagataella, Lindnera, Ogataea, Saccharomyces, Schizosaccharomyces, Wickerhamomyces, Xanthophyllomyces and Yarrowia, Micrococcus preferably Cutibacterium acnes, Lactococcus lactis, Lactobacillus rhamnosus, Lactobacillus crispatus, Lactobacillus gasseri, Lactobacillus plantarum; Lactobacillus delbrickii, Lactobacillus reuteri, Lactobacillus paracasei, Lactobacillus fermentum, Staph. epidermidis, Bacillus subtilis, Bacillus megaterium, Micrococcus luteus, Micrococcus lylae, Micrococcus antarcticus, Micrococcus endophyticus, Micrococcus flavus, Micrococcus terreus, Micrococcus yunnanensis, Arthrobacter agilis, Nesterenkonia halobia, Kocuria kristinae, Kocuria rosea, Kocuria varians, Kytococcus sedentarius, Dermacoccus nishinomiyaensis, and mixtures thereof.

13. The method of claim 1, wherein the probiotic microorganism is selected from the group consisting of S. epidermidis, L. fermentum, DSM 32609 L. rhamnosus, DSM 32758 L. plantarum, DSM 32749 L. delbrueckii susp. bulgaricus, DSM 33370 L. plantarum LN5, DSM 33377 L. brevis LN32, DSM 33368 L. plantarum S3, DSM 33369 L. plantarum S11, DSM 33376 L. paracasei S20, DSM 33375 L. paracasei S23, DSM 33374 L. reuteri F12, DSM 33367 L. plantarum F8, DSM 33366 L. plantarum S4, DSM 33364 L. plantarum S28, DSM 33363 L. plantarum S27, DSM 33373 L. paracasei S18a, DSM 33365 L. plantarum S18b, DSM 33362 L. plantarum S13, DSM 32767 Lactococcus lactis sups. lactis, L. fermentum DSM 32750, Propionibacterium acnes, and Cutibacterium acnes.

14. The method of claim 1, further comprising:
loading the multiphase biomaterial with at least one ingredient or nutrient before or after or in parallel to loading the multiphase biomaterial with the microorganisms,
wherein the at least one ingredient or nutrient is selected from the group consisting of amino acids, fatty acid salts, anthocyanins, monosaccharides, and extracts.

15. A non-woven bacterially synthesized nanocellulose (BNC) multiphase biomaterial comprising at least two different bacterial cellulose networks comprising at least one living microorganism obtained by the method of claim 1.

16. The BNC multiphase biomaterial of claim 15, comprising at least one living microorganism at a concentration of at least 3.00×10⁷ cells of microorganism per gram of cellulose.

17. (canceled)
